(19) United States
(12) Patent Application Publication
(10) Pub. No.: US 2002/0131960 A1
(43) Pub. Date: Sep. 19, 2002

(54) Artificial antigen presenting cells and methods of use thereof

(76) Inventors: Michel Sadelain, New York, NY (US); Jean Baptiste Latouche, New York, NY (US)

Correspondence Address: FROMMER LAWRENCE & HAUG, 745 FIFTH AVENUE- 10TH FL., NEW YORK, NY, 10151, US

(21) Appl. No.: 09/872,832

(22) Filed: Jun. 1, 2001

Related U.S. Application Data
(60) Provisional application No. 60/209,157, filed on Jun. 2, 2000.

(51) Int. Cl. A61K 48/00; G01N 33/567; C12N 15/86; C12N 5/08

(52) U.S. Cl. 424/93.21; 435/7.21; 435/456; 435/372

(57) ABSTRACT

The invention provides an artificial antigen presenting cell (AAPC) comprising a eukaryotic cell expressing an antigen presenting complex comprising a human leukocyte antigen (HLA) molecule of a single type, at least one exogenous accessory molecule and at least one exogenous T cell-specific epitope. Methods of use for activation of T lymphocytes are also provided.

### CROSS-REFERENCE TO RELATED APPLICATIONS

This application claims priority to and benefit of U.S. Patent application serial no. 60/209,157, filed Jun. 2, 2000.

### STATEMENT OF RIGHTS TO INVENTIONS MADE UNDER FEDERALLY SPONSORED RESEARCH

This work was supported by grant CA-59350, the Dewitt Wallace Fund, and the McDonnell Scholars Award for Molecular Medicine. The United States Government may have certain rights to the invention.

### FIELD OF THE INVENTION

This invention relates to the adoptive transfer of antigen-specific cytotoxic T lymphocytes (CTLs) as a therapeutic approach for a number of diseases. Stable artificial antigen-presenting cells (AAPCs) that can be used to stimulate T cells of any patient of a given human leukocyte antigen (HLA) type have been made. Mouse fibroblasts were retrovirally transduced with a single HLA-peptide complex along with the human accessory molecules B7.1, ICAM-1, and LFA-3. These AAPCs consistently elicit strong stimulation and expansion of HLA-restricted CTLs. Owing to the high efficiency of retrovirus-mediated gene transfer, stable AAPCs are readily engineered for any HLA molecule and any specific peptide.

### BACKGROUND

Mammalian hematopoietic (blood) cells provide a diverse range of physiologic activities. Hematopoietic cells are divided into lymphoid, myeloid and erythroid lineages. The lymphoid lineage, comprising B, T and natural killer (NK) cells, provides for the production of antibodies, regulation of the cellular immune system, detection of foreign agents in the blood, detection of cells foreign to the host, and the like. The myeloid lineage, which includes monocytes, granulocytes, megakaryocytes, as well as other cells, monitors for the presence of foreign bodies, provides protection against neoplastic cells, scavenges foreign materials, produces platelets, and the like. The erythroid lineage provides the red blood cells, which act as oxygen carriers.

Hematopoietic cells are identifiable by the presence of a variety of cell surface protein “markers.” Such markers may be either specific to a particular lineage or be present on more than one cell type. The markers also change with stages of differentiation. Miltenyi Biotec GmbH supplies high gradient magnetic separation devices suitable for use in cell purification.

Lymphocytes (B and T cells) are highly specialized hematopoietic cells. During the development of the B and T cell lineages, phenotypic and molecular differentiation of primitive cells leads to mature stages where rearrangement of the lymphocyte antigen receptors occur, namely the immunoglobulin (Ig) or T cell receptor (TCR) chains. Van Noesel et al. (1993) Blood 82:363-373; and Godfrey and Zlotnik (1993) Immunol. Today 14:547-553. Commitment to the B cell lineage, expression of the B cell receptor complex and Ig gene rearrangements take place in the bone marrow or fetal liver. Uckun (1990) Blood 76:1908-1923; and Li et al. (1993) J. Exp. Med. 178:951-960.

Unlike B cell differentiation, T cell development requires passage of T-progenitor cells through the thymus gland to achieve efficient TCR rearrangement and major histocompatibility complex (MHC)-restriction. At the thymic stage, immature T cells are called thymocytes. The intrathymic stages of T cell development have been extensively studied in mice and to a lesser extent in man. Godfrey and Zlotnik (1993); Galy et al. (1993) J. Exp. Med. 178:391-401; Terstappen et al. (1992) Blood 79:666-677; and Sanchez et al. (1993) J. Exp. Med. 178:1857-1866. Studies in animals using mice or quail/chick chimeras and studies in man with constructs of fetal liver and thymus implanted into surrogate severe combined immunodeficiency (SCID) mice, have shown that a constant input of hematopoietic cells is needed to sustain thymopoiesis. Le Douarin et al. (1973) Nature New Biol. 246:25-27; Scollay et al. (1986) Immunol. Rev. 91:129-157; and McCune et al. (1988) Science 241:1632-1639.

MHC products are grouped into three major classes, referred to as I, II, and III. T cells that serve mainly as helper cells express CD4 and primarily interact with Class II molecules, whereas CD8-expressing cells, which mostly represent cytotoxic effector cells interact with Class I molecules.

Class I molecules are membrane glycoproteins with the ability to bind peptides derived primarily from intracellular degradation of endogenous proteins. Table 1 provides a number of these peptides. As shown in FIG. 1, complexes of MHC molecules with peptides derived from viral, bacterial and other foreign proteins comprise the ligand that triggers the antigen responsiveness of T cells. In contrast, complexes of MHC molecules with peptides derived from normal cellular products play a role in “teaching” T cells to tolerate self-peptides in the thymus. Class I molecules do not present entire, intact antigens; rather, the present peptide fragments thereof, “loaded” onto their peptide-binding groove. Several artificial loading systems have been described. WO 96/27392; Schultze et al. (1997) J. Clin. Invest. 100:2757-2765; and Sprent et al. (1997) In, Dendritic Cells in Fundamental and Clinical Immunology, Ricciardi-Castagnoli ed., Plenum Press, New York.

TABLE 1

| HLA | Peptide | Source | SEQ ID NO: |
|---|---|---|---|
| A1 | Y T S D Y F I S Y | Ets-1 | 1 |
| | Y L D D P D L K Y | Cytosine methyl transferase | 2 |
| | I A D M G H L K Y | Nuclearfactor | 3 |
| | S T D H I P I L Y | Fructose-6-amino transferase | 4 |
| | D S D G S F F L Y | Human IgG₄ 279-287 | 5 |
| | A T D F K F A M Y | Cyclin Type D | 6 |
| | Y T A V V P L V Y | Human J-chain 102-110 | 7 |
| | Y T D Y G G L I F N S Y | Cytochrome C oxidase | 8 |
| A2.1 | L L D V P T A A V | IP-30 signal sequence | 9 |
| | S L L P A I V E L | Protein phosphate 2A | 10 |
| | Y L L P A I V E I | ATP-dep. RNA helicase | 11 |
| | M V D G T L L L L | HLA-E signal sequence | 12 |
| | Y M N G T M S Q V | Tyrosinase | 13 |
| | M L L S V P L L L G | Calreticulin signal sequence | 14 |
| | L L L D V P T A A V | IP-30 signal sequence | 15 |
| | L L L D V P T A A V Q A | IP-30 signal sequence | 16 |
| | V L F R G G P R G L L A V A | SSRα signal sequence | 17 |
| A11 | S V L N L V I V K | Ribosomal protein S6 | 18 |
| | K V V N P L F E K | Ribosomal protein L7A | 19 |
| | R T Q N V L G E K | Ribosomal protein S3 | 20 |
| | A S F D K A K L K | Thymosin B-10 | 21 |
| | A T A G D G X X E L R K | Prohibitin 22 | |
| A24 | K Y P N E F F L L | Protein phosphatase 1 | 23 |
| | Y Y E E Q H P E L | NK/T-cell activation protein | 24 |
| | A Y V H M V T H F | Unknown | 25 |
| | V Y X K H P V S X | Unknown | 26 |
| A68.1 | D V F R D P A L K | Ribosomal 60S homologue | 27 |
| | K T G G P I Y K R | Influenza NP 91-99 | 28 |
| | T V F D A K R L I G R | HSP70 protein B/HSP70 | 29 |
| B7 | A P R T V A L T A | HLA-DP signal sequence | 30 |
| | A P R T L V L L L | HLA-A2.1 signal sequence | 31 |
| | A P R P P P K P M | Ribosomal S26 protein | 32 |
| | S P R Y I F T M L | Topoisomerase II | 33 |
| | R P K S N J V L L | CD20 | 34 |
| | L V M A P R T V L | HLA-B7 signal sequence | 35 |
| | A P R T V A L T A L | HLA-DP signal sequence | 36 |
| | A A S K E R S G V S L | histone H1 | 37 |
| B27 | R R I K E I V K K | HSP89α | 38 |
| | G R I D K P I L K | Ribosome protein | 39 |
| | R R S K E I T V R | ATP-dep. RNA helicase | 40 |
| | R R V K E V V K K | HSP89β | 41 |
| | R R Y Q K S T W L | Histone H3.3 | 42 |

MHC polymorphism is notable in two respects; its extent and its nature. The usual situation with polymorphic loci is that there are one or two alleles that occur at high frequencies and a few additional alleles that occur at much lower frequencies. At the latest count, 59, 118 and 36 alleles have registered at the HLA-A, -B and -C loci, respectively; for the HLA-DRB 1, DQA1,-DQB 1 and -DPA1 loci the numbers are 168, 19, 30, 73 and 8, respectively. While a few of these alleles may represent rare variants, most are known to occur at appreciable frequencies. Moreover, new alleles are still being described and only very few human populations have been HLA-typed adequately.

Proteasomes, process proteins found in the cytosol into short peptides. Proteasomes do not distinguish between self and non-self proteins and normally act on the cell's own proteins that have, for one reason or another, been marked for disposal. In an infected cell, however, proteasomes also slice viral proteins into peptides. The various peptides are then transported across the membranes of the rough endoplasmic reticulum (RER). The transport is effected by a set of specialized protein structures residing in the RER membrane, the peptide transporters. On the luminal side of the membrane, the peptides are loaded onto MHC-I molecules. A cell possesses different types of proteasomes and a variety of peptide transporters. Those involved in the generation of peptides destined to be loaded onto MHC-I molecules are referred to as low molecular weight (mass) proteins or large multifunctional protease (both abbreviated as LMP) and transporters associated with antigen process (TAP, a member of a family of ATP-binding cassette (ABC) transporters).

The MHC class I molecules consist of two polypeptide chains, one of which is β2-microglobulin. The chains are synthesized separately on the luminal surface of the RER and when they come together to form a dimer, the peptides are loaded onto them, into a specialized groove formed by the a chain. The loaded MHC class I molecules are then transported, via the Golgi apparatus and with the help of transport and exocytic vesicles, to the cell surface where they are integrated into the plasma membrane. The cell's surface is thus studded by MHC class I molecules complexed with peptides. In an uninfected cell, the molecules are loaded with self peptides; in a virally infected cell, many of them bear non-self (viral) peptides. The adaptive immune system has learned to ignore the MHC-self peptide complexes and to respond to the non-self-peptide-MHC assemblies. The latter are recognized by the CD8⁺ T lymphocyte T cell receptors (TCRs), and this recognition activates the T cells. The activated cells divide and some of their progeny differentiate into lymphocytes capable of killing cells that display the same peptide, or highly related, so-called heteroclytic peptides, on their class I MHC molecules. These CTLs target virus-infected cells, or tumor cells, depending on the peptide, and eliminate them.

The generation of peptides from antigenic proteins is “antigen processing”; the display of the MHC-peptide complexes at the cell surface as antigen presentation; the cells that carry out the latter are known as antigen presenting cells (APCs).

The definitive T cell marker is the TCR. There are presently two defined types of TCR. TCR-2 is a heterodimer of two disulfide-linked transmembrane polypeptides (cc and β), TCR-1 is structurally similar but consists of γ and δ polypeptides. The α and β or γ and δ polypeptides form a heterodimer which contains an antigen recognition site. These heterodimers recognize antigen in association with MHC on the surface of APC. All of these proteins contain a variable region that contributes to the antigen recognition site and a constant region that forms the bulk of the molecule and includes the transmembrane region and cytoplasmic tail. Both receptors are associated with a complex of polypeptides making up the CD3 complex. The CD3 complex comprises the δ, ε and γ transmembrane polypeptides. The CD3 complex mediates signal transduction when T cells are activated by antigen binding to the TCR.

Approximately 95% of blood T cells express TCR-2 and up to 5% have TCR-1. The TCR-2 bearing cells can be subdivided further into two distinct non-overlapping populations. CD4⁺ T cells which generally recognize antigens in association with MHC class II, and CD8⁺ T cells which recognize antigens in association with MHC class I.

Dendritic cells (DCs) are APCs that are essential for initiation of primary immune responses and the development of tolerance. DCs express MHC, necessary for stimulation of naive T cell populations. The hematopoietic development of DCs is distinct and may follow several precursor pathways, some of which are closely linked to monocytes. See, for review, Avigan (1999) Blood Rev. 13:51-64. Different DC subsets have distinct developmental pathways. The emerging concept is that one DC subset has regulatory functions that may contribute to the induction of tolerance to self-antigens. Austyn (1998) Curr. Opin. Hematol. 5:3-15. Conversely, DCs, or a subset thereof, may also be involved in the induction of autoimmunity, the immune responses to self-proteins. It is thought that certain autoimmune responses may be due to micro-environmental tissue injury followed by local DC activation and subsequent interaction with T cells to initiate the immune response. Ibrahim et al. (1995) Immunol. Today 16:181-186.

The ability of DCs to initiate T cell responses is being used in cancer vaccines. For instance, DCs are isolated from CD34⁺ cells or monocytes, pulsed with tumor-derived peptides or proteins and returned to the patient to act as APCs in cancer-specific T cell induction. Brugger et al. (1999) Ann. N.Y. Acad. Sci. 872:363-371. Animal models have demonstrated that DC tumor vaccines reverse T cell anergy and result in subsequent tumor rejection. Avigan (1999); see also, Tarte et al. (1999) Leukemia 113:653-663; Colaco (1999) Molec. Med. Today 5:14-17; Timmerman et al. (1999) Ann. Rev. Med. 50:507-529; Hart et al. (1999) Semin. Hematol. 36:21-25; Thurnher et al. (1998) Urol. Int. 61:67-71; and Hermans et al. (1998) N.Z. Med. J. 111:111-113. DCs have been proposed for use as adjuvants in vaccination and in recombinant vaccines. Fernandez et al. (1998) Cyto. Cell. Mol. Ther. 4:53-65; and Gilboa et al. (1998) Cancer Immunol. Immunother. 46:82-87.

Several distinct signals contribute to effectively initiate and sustain T cell activation and proliferation. The T cell receptor must engage the MHC-peptide complex, which provides the basis for antigen specificity. Davis et al. (1993) Curr. Opin. Immunol. 5:45-49. Signaling through the CD28 receptor provides a powerful costimulatory signal following engagement of the B7.1 (CD80) or B7.2 (CD86) ligand. Lenschow et al. (1996) Annu. Rev. Immunol. 14:233-258. The adhesion molecule ICAM-1 (CD54) provides a synergistic signal through the LFA-1 (CD11/CD18) molecule expressed on T cells, whereas other molecules, in particular LFA-3 (CD58), ligand of the T cell molecule CD2, can also mediate costimulatory as well as adhesion functions. Shaw et al. (1997) Immunity 6:361-369; and Watts et al. (1999) Curr. Opin. Immunol. 11:286-293. These accessory molecules are expressed at high levels on DCs, which are able to induce naive T lymphocytes, and a major role of B7.1, ICAM-1, and LFA-3 in costimulating CTLs has been reported. Banchereau et al. (1998); Parra et al. (1997) J. Immunol. 158:637-642; Fields et al. (1998) J. Immunol. 161:5268-5275; and Deeths and Mescher (1999) Eur. J. Immunol. 29:45-53. mAb specific for human DC are described in WO 010/117687.

The infusion of antigen-specific T lymphocytes is a potential therapy against certain cancers and infectious diseases. Rosenberg (1991) Cancer Res. 51 :5074s-5079s; Melief and Kast (1995) Immunol. Rev. 145:167-177; Riddell and Greenberg (1995) Annu. Rev. Immunol. 13:545-586; Rooney et al. (1998) Vox Sang. 2:497-498; and O'Reilly et al. (1998) Springer Semin. Immunopathol. 20:455-491. One limitation to its broad usage is the generation of autologous T cells directed against well-defined epitopes. The induction and expansion of antigen-specific T cells require a suitable source and amount of APCs such as DCs, optimal antigen presentation and T cell costimulation. Lanzavecchia et al. (1999) Cell 96:1-4; and Dustin and Shaw (1999) Science 283:649-650. These requirements can be met by APCs such as Epstein-Barr virus-transformed B cells and DCs, which constitutively express high levels of costimulatory, adhesion, and MHC molecules. Banchereau et al. (1998) Nature 392:245-252; and Grakoui et al. (1999) Science 285:221-227. An APC based on Drosophila cells has been described. WO 96/27392.

Studies on and therapeutic use of DCs have been hampered by scarcity of the cells and the relative lack of DC-specific cell surface markers. Methods for DC isolation are based on either maturational changes after a short culture period, like the acquisition of low buoyant density or the expression of DC activation/maturation antigens (CD83, CMRF-44 and CMRF-56). Young et al. (1988) Cell Immunol. 111:167; Van Voorhis et al. (1982) J. Exp. Med. 155:1172; Zhou et al. (1995) J. Immunol. 154:3821-3835; Feamley et al. (1997) Blood 89:3708-3716; Mannering et al. (1988) J. Immunol. Met. 219:69-83; Hock et al. (1999) Tiss. Antigens 53:320-334; and Hock et al. Immunol. 83:573-581.

Despite a cumbersome generation process, the use of autologous cells to present well-defined epitopes is mandated to obviate strong allogeneic responses that would unavoidably develop if allogeneic DCs or EBV-transformed B cells were used as the APCs. This limits the ability to provide therapeutically effective APCs.

### OBJECTS AND SUMMARY OF THE INVENTION

The invention encompasses a parental AAPC comprising a eukaryotic cell expressing β2-microglobulin and at least one exogenous accessory molecule.

The invention further encompasses an MHC-specific parental AAPC comprising a eukaryotic cell expressing β2-microglobulin, at least one exogenous accessory molecule and a HLA molecule of a single type.

The invention further encompasses an AAPC comprising a eukaryotic cell expressing an antigen presenting complex comprising β2-microglobulin, at least one exogenous accessory molecule, a HLA molecule of a single type and presenting at least one exogenous T cell-specific epitope. Methods of treatment utilizing the AAPC are also encompassed by the invention.

The invention encompasses a method of activating CTLs by obtaining an AAPC; obtaining a suitable population of T lymphocytes; contacting the AAPC with the population of T lymphocytes under conditions suitable for T lymphocyte activation; and isolating the activated CTLs. Compositions of activated CTLs obtained by the method are also encompassed by the invention as are methods of treatment using the cells.

The invention also provides a method of screening for accessory molecules by obtaining an AAPC; expressing genes encoding potential accessory molecules in the AAPC; obtaining a control AAPC that does not express potential accessory molecules; obtaining a suitable population of T lymphocytes; contacting the T lymphocytes with the AAPC under conditions suitable for activating T lymphocytes; contacting the T lymphocytes with the control AAPC under conditions suitable for activating T lymphocytes; and comparing the activation of the T lymphocytes to the activation of the T lymphocytes from the control sample; wherein, if the activation of the T lymphocytes is greater than that of the T lymphocytes from the control sample, the potential accessory molecule is designated an accessory molecule.

The invention further encompasses a method of screening for T cell-specific antigens by obtaining an MHC-specific parental AAPC; allowing the MHC-specific parental AAPC to present potential T cell specific antigens; obtaining a control AAPC that does not present potential T cell specific antigens; obtaining a suitable population of T lymphocytes; contacting the T lymphocytes with the AAPC under conditions suitable for activating T lymphocytes; contacting the T lymphocytes with the control AAPC under conditions suitable for activating T lymphocytes; and comparing the activation of the T lymphocytes to the activation of the T lymphocytes from the control; wherein, if the activation of the T lymphocytes is greater than that of the T lymphocytes of the control, the potential T cell specific antigens is designated a T cell specific antigen.

The invention further provides a method of identifying, within a test population of CTLs, CTL specifically activated against a known T cell antigen by obtaining an AAPC; allowing the AAPC to present the known T cell antigen; obtaining a control AAPC that does not present the known T cell antigen; obtaining the test population of T lymphocytes; contacting the test population of T lymphocytes with the AAPC under conditions suitable for activating T lymphocytes; contacting the T lymphocytes with the control AAPC under conditions suitable for activating T lymphocytes; and comparing the activation of the T lymphocytes to the activation of the T lymphocytes from the control; wherein, if the activation of the T lymphocytes is greater than that of the T lymphocytes of the control, the potential accessory molecule is designated an accessory molecule.

### BRIEF DESCRIPTION OF THE DRAWINGS

FIG. 1 is a schematic showing T cell activation.

FIGS. 2A and 2B provide schematic diagrams of the recombinant molecules described herein.

FIG. 2C is a series of graphs depicting flow cytometry analysis of HLA A2.1, CD80, CD54, and CD58 expression in AAPCs.

FIG. 3A is a set of graphs depicting cytotoxicity of T cells from HLA A2.1⁺ donor stimulated with primary autologous dendritic cells (left panel) of AAPC^{A2F} (right panel).

FIG. 3B depicts the results of flow cytometry analysis of CD8⁺ T cells before (upper panels) and after (lower panels) cocultivation with HLA A2.1⁺ AAPCs encoding the flu peptide.

FIG. 4 is a bar graph depicting expansion of primary CD8⁺ T cells stimulated with AAPC^{A2F} or flu peptide—pulsed autologous dendritic cells.

FIG. 5 is a series of graphs showing that AAPCs induce cytotoxic T cell responses against tumor antigens. Filled symbols are target cells pulsed with the relevant peptide and open symbols correspond to target cells pulsed with an irrelevant peptide.

FIG. 6 is a series of graphs depicting cytotoxic T lymphocyte induction against different tumor antigens in different HLA A2.1⁺ donors. T cells purified from three HLA A2.1⁺ donors (A, B, C) were stimulated twice by AAPCA2F, AAPCA2G, or AAPCA2M.

FIG. 7 is a series of graphs depicting HLA restricted cytolysis of melanoma cells by CTLs induced by AAPC^{A2G} and AAPC^{A2M} .

FIG. 8 illustrates the cytotoxicity results obtained with EBV/LMP1.1 peptide.

FIG. 9 is a bar graph depicting the results of an ELISpot assay of AAPC-flu-induced IFN-γ production.

FIG. 10 is a schematic depicting tetrameric complexes that allow detection of specific CTLs by flow cytometry.

FIG. 11 shows detection of specific CTLs in cytotoxicity assays (A) or by flow cytometry using HLA class I/peptide tetrameric complexes (B).

FIG. 12 shows detection of specific CTLs by flow cytometry using HLA class I/peptide tetrameric complexes after coculture of T cells from HLA A2.1⁺ donor with different AAPCs or autologous EBV-transformed B cells.

FIG. 13 shows CTLs stimulated by autologous EBV-transformed B cells or AAPCs encoding the LMP 1.1 peptide (AAPC^{A2L}) were compared in their abilities to kill different tumor cell lines.

FIG. 13A shows stimulation with autologous EBV BLCL.

FIG. 13B shows stimulation with AAPC^{A2L}.

FIG. 14 is a graph depicting CTL activation, determined by ⁵¹Cr release by AAPC expressing a peptide antigen (495) or an entire protein (pp65).

In FIG. 14, ♦ represents E495/T495; ▪ represents Epp65/T495; Δ represents E495/T120; and * represents Epp65/Tflu.

FIG. 15 shows induction of Wilm's tumor gene (WT1) specific CTLs.

FIGS. 15A and B show WT1 tetramer staining of (A) CTLs stimulated on WT1 (Db126) AAPCs and (B) negative control, CTLs stimulated on WT1 (Wh187) AAPCs.

FIG. 15C shows the results of the ⁵¹Cr release assay (T2 cells).

In FIG. 15C, ♦ represents Db126 TL/T2-Db126 and ▪ represents Db126 TL/T2-Wh187.

FIG. 16 shows induction of human Telomerase reverse transcriptase (hTERT) specific CTLs.

FIGS. 16A and B show hTERT (p865) tetramer staining of (A) CTLs stimulated on hTERT (p865) AAPCs and (B) negative control, CTLs stimulated on hTERT (p865) AAPCs.

FIG. 16C shows the results of the ⁵¹Cr release assay (T2 cells). In FIG. 16C, ♦ represents P865 TL/T2-P865; ▪ represents Flu TL/T2- P865; ▴ represents P865 TL/T2-P540; and * represents Flu TL/T2-P540.

FIG. 17 shows the results of a 51Cr release assay of specific killing of HLA A2.1⁺ tumor cell line SKLY by hTERT specific CTL. In FIG. 17, ♦ represents P865 TL/SKLY and ▪ represents Flu TL/SKLY.

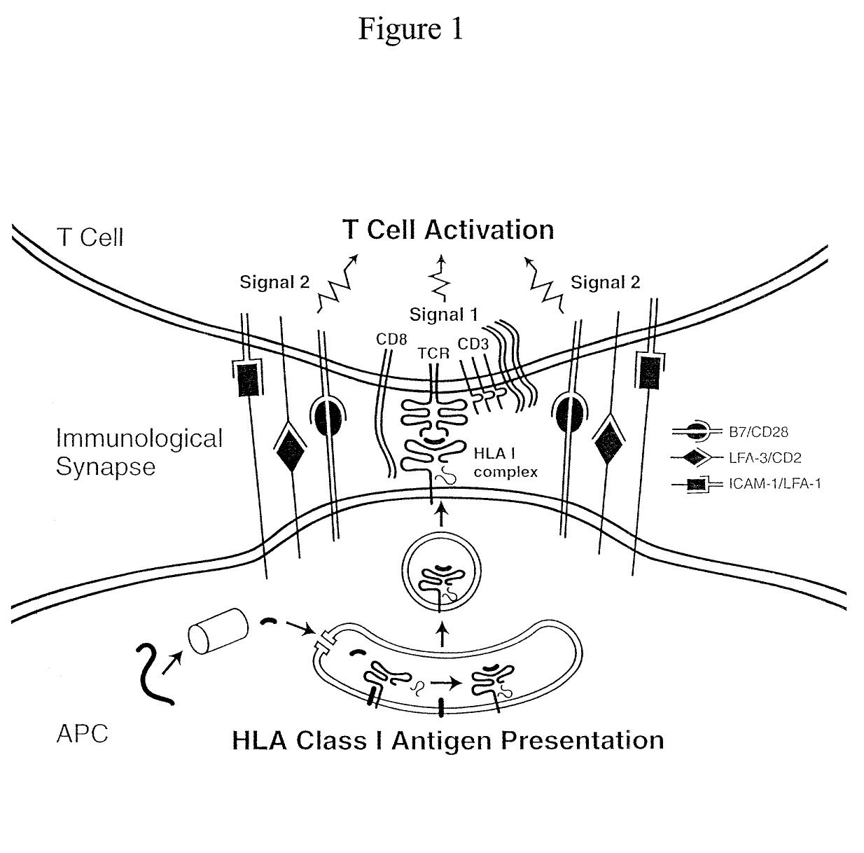

### DETAILED DESCRIPTION

Following the methods described herein, the examples demonstrate potent induction and expansion of CTLs against viral and self-peptides presented by AAPC in the context of a specific HLA.

Three human costimulatory and adhesion molecules, B7.1, ICAM-1 and LFA-3, were retrovirally transduced in xenogeneic mouse fibroblasts with a single HLA molecule. To efficiently present MHC-peptide complexes to CTLs, single MHC class I molecules were coexpressed with human β2-microglobulin and a single genetically encoded peptide. Starting from peripheral blood T cells harvested from HLA A2.1⁺ donors, potent induction and expansion of CTLs against viral and self peptides presented in the context of HLA A2.1 is demonstrated herein. Three epitopes derived from influenza matrix, MART-1, gp 100, and LMP-1 proteins were investigated. Bednarek et al. (1991) J. Immunol. 147:40474053% Morrison et al. (1992) Eur. J. Immunol. 22:903-907; Kawakami et al. (1994) J. Exp. Med. 180:347-352; and Parkhurst et al. (1996) J. Immunol. 157:2539-2548. Cytotoxicity was highly specific and increased by restimulation with the AAPCs. CTL induction was more efficient than that obtained with autologous blood-derived DCs. Cytotoxic activity induced by AAPCs encoding the MART-1 or gp 100-derived peptide was elevated against HLA A2.1⁺ (but not A2.1⁻) melanoma cell lines that express these antigens. These findings establish that high level cell-surface expression of B7.1, ICAM-1, LFA-3 and single MHC class I-peptide complexes is sufficient to effectively induce strong antigen-specific CTL responses in human peripheral blood cells. Such AAPCs are extremely valuable for the investigation of primary T cell activation and the use of antigen-specific T cells for adoptive cell therapies and diagnostics.

The invention encompasses a parental AAPC comprising a eukaryotic cell expressing β2-microglobulin and at least one exogenous accessory molecule.

The invention further encompasses an MHC-specific parental AAPC comprising a eukaryotic cell expressing β2-microglobulin, at least one exogenous accessory molecule and a human leukocyte antigen (HLA) molecule of a single type.

The invention further encompasses an AAPC comprising a eukaryotic cell expressing an antigen presenting complex comprising β2-microglobulin, at least one exogenous accessory molecule, a human leukocyte antigen (HLA) molecule of a single type and presenting at least one exogenous T cell-specific epitope. Methods of treatment utilizing the AAPC are also encompassed by the invention.

The cells used to make parental AAPC and AAPC can be human, murine, rodentia, insect, or any other mammalian cells. The cells can be human but it is not necessary. In fact, the use of non-human cells can increase the activity of the cells by decreasing non-specific (background) antigen presentation. The cells can be autologous or non-autologous. The cells can be fibroblasts, T lymphocytes, tumor cells, a transformed cell line, cells of hematopoietic origin, keratinocyte muscle cells or stromal cells. Preferably, the cells are fibroblasts.

The β2 microglobulin can be endogenous or exogenous. Preferably, the β2 microglobulin is human P2 microglobulin.

The accessory molecule is selected from the group consisting of B7.1, B7.2, ICAM-1, LFA-3, CD40, CD40L, SLAM and 41BB ligand. Preferably, the accessory molecule is B7.1. preferably, the accessory molecule is ICAM-1. Even more preferably, the accessory molecules are B7.1 and ICAM-1.

The HLA molecule can be endogenous or exogenous. Preferably, the HLA molecule type is HLA-I. The HLA-1 can be A2.1, or any other HLA A, B or C.

The exogenous T cell specific epitope can be one or more antigens. The epitope can be derived from a peptide specific to a tumor cell, a bacterial cell, a virus, a parasite or a normal human cell. The T cell-specific epitope can be derived from a peptide that is a mutant or enhanced peptide derived from naturally occurring peptide specific to a tumor cell, a bacterial cell, a virus, a parasite or a human cell.

The HLA can be A1 and the T cell specific epitope can be YTSDYFISY, YLDDPDLKY, IADMGHLKY, STDHIPILY, DSDGSFFLY, ATDFKFAMY, YTAVVPLVY and YTDYGGLIFNSY.

The HLA can be A2.1 and the T cell specific epitope can be LLDVPTAAV, SLLPAIVEL, YLLPAIVEI, MVDGTLLLL, YMNGTMSQV, MLLSVPLLLG, LLLDVPTAAV, LLLDVPTAAVQA, and VLFRGGPRGLLAVA.

The HLA can be A11 and the T cell specific epitope can be SVLNLVIVK, KVVNPLFEK, RTQNVLGEK, ASFDKAKLK, and ATAGDGXXELRK.

The HLA can be A24 and the T cell specific epitope can be KYPNEFFLL, YYEEQHPEL, AYVHMVTHF, and VYXKHPVSX.

The HLA can be A68.1 and the T cell specific epitope can be DVFRDPALK, KTGGPIYKR, and TVFDAKRLIGR.

The HLA can be B7 and the T cell specific epitope can be APRTVALTA, APRTLVLLL, APRPPPKPM, SPRYIFTML, RPKSNIVLL, LVMAPRTVL, APRTVALTAL, and AASKERSGVSL.

The HLA can be B27 and the T cell specific epitope can be RRIKEIVKK, GRIDKPILK, RRSKEITVR, RRVKEVVKK, and RRYQKSTWL.

The T cell-specific epitope can be influenza matrix, Mart-1, gp100, LMP-1, Wt-1, acid phosphatase, Her-2/neu and telomerase.

Preferably, the β2-microglobulin and the accessory molecule and the HLA molecule are expressed from genes introduced into the cell by a recombinant virus. The T cell specific epitope can be expressed from genes introduced into the cell by a recombinant virus, or is loaded onto the cell.

The AAPC can further contain alterations either by mutation or gene fusion. The alterations can be to endogenous genes or to the introduced genes. Such alterations include, but are not limited to, those that decrease endogenous peptide transport so as to enhance presentation of the exogenous molecules, those that increase antigen processing and those that increase antigenicity of the antigen.

The invention encompasses a method of activating CTLs by obtaining an AAPC; obtaining a suitable population of T lymphocytes; contacting the AAPC with the population of T lymphocytes under conditions suitable for T lymphocyte activation; and isolating the activated CTLs. Compositions of activated CTLs obtained by the method are also encompassed by the invention as are methods of treatment utilizing the cells. The CTLs can be restimulated by contacting again with the AAPC. There can be second, third, fourth, etc. restimulations by contact with the AAPC.

The invention also provides a method of screening for accessory molecules by obtaining an AAPC; expressing genes encoding potential accessory molecules in the AAPC; obtaining a control AAPC that does not express potential accessory molecules; obtaining a suitable population of T lymphocytes; contacting the T lymphocytes with the AAPC under conditions suitable for activating T lymphocytes; contacting the T lymphocytes with the control AAPC under conditions suitable for activating T lymphocytes; and comparing the activation of the T lymphocytes to the activation of the T lymphocytes from the control sample; wherein, if the activation of the T lymphocytes is greater than that of the T lymphocytes from the control sample, the potential accessory molecule is designated an accessory molecule.

The invention further encompasses a method of screening for T cell-specific antigens by obtaining an MHC-specific parental AAPC; allowing the MHC-specific parental AAPC to present potential T cell specific antigens; obtaining a control AAPC that does not present potential T cell specific antigens; obtaining a suitable population of T lymphocytes; contacting the T lymphocytes with the AAPC under conditions suitable for activating T lymphocytes; contacting the T lymphocytes with the control AAPC under conditions suitable for activating T lymphocytes; and comparing the activation of the T lymphocytes to the activation of the T lymphocytes from the control; wherein, if the activation of the T lymphocytes is greater than that of the T lymphocytes of the control, the potential T cell specific antigens is designated a T cell specific antigen.

The potential T cell specific epitope can be produced by any method known in the art including, but not limited to recombinatorial chemistry and a phage display library.

The invention further provides a method of identifying, within a test population of CTLs, CTLs specifically activated against a known T cell antigen by obtaining an AAPC; allowing the AAPC to present the known T cell antigen; obtaining a control AAPC that does not present the known T cell antigen; obtaining the test population of T lymphocytes; contacting the test population of T lymphocytes with the AAPC under conditions suitable for activating T lymphocytes; contacting the T lymphocytes with the control AAPC under conditions suitable for activating T lymphocytes; and comparing the activation of the T lymphocytes to the activation of the T lymphocytes from the control; wherein, if the activation of the T lymphocytes is greater than that of the T lymphocytes of the control, the potential accessory molecule is designated an accessory molecule. Activation can be measured by any method known in the art including, but not limited to, cytokine secretion and measuring a T cell surface marker. The cytokine assayed can be any known in the art including, but not limited to, IFN-γ, IL-4, IL-10 or TNF. The T cell surface marker can be any known in the art including, but not limited to, an activation marker and effector molecule. Suitable activation markers include, but are not limited to, CD69, IL-2 receptor and IL-15 receptor. Suitable effector molecules include, but are not limited to, FasL and trail.

Cytokine secretion can be measured by immunologic methods such as by the enzyme-linked immunospot (ELISpot) assay. ELISpot was originally developed for the detection of individual B cells secreting antigen-specific antibodies. This method has since been adapted for the detection of individual cells secreting specific cytokines or other antigens. For instance, a multitest plate is coated with antibodies against IFN-γ is incubated with peripheral blood lymphocytes and an antigen/mitogen to activate the CTLs. During incubation IFN-γ secretion will occur in antigen stimulated cells. After incubation cells are removed by washing, and a detection system localizes the antibody bound IFN-γ. Each spot represents the “footprint” of a IFN-γ producing cell. This method quantifies the number of cells stimulated by a specific antigen.

Identification of activated CTLs can also be used to measure the proportion of activated CTLs in the test population of CTLs. This can be important for certain diagnostic purposes when identification alone is insufficient.

Other uses of AAPCs include, but are not limited to, investigation of primary T cell activation, and diagnostic applications. Primary T cell activation allows discovery of antigens and accessory molecules. Diagnostic applications include, but are not limited to, cell-based assays for quantifying immune responses in normal, infected or treated (vaccinated) patients.

Any suitable antigenic peptide is suitable for use herein. Sources of antigen include, but are not limited to parasitic, bacterial, viral, cancer, tissues, and tolerogenic proteins. The antigen can be expressed as a peptide or as an intact protein or portion thereof. The intact protein or a portion thereof can be native or mutagenized. It has now been shown that the intact protein is processed by the AAPC for proper presentation. Suitable peptides include, but are not limited to, those listed in Table 1, WT-1, acid phosphates peptide, Her-2/neu and telomerase in addition to those described herein.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. mAbs are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Preferably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Preferably, the cells are collected in a medium comprising 2% fetal calf serum (FCS) or 0.2% bovine serum albumin (BSA) or any other suitable, preferably sterile, isotonic medium.

Genetic modification of the AAPCs can be accomplished at any point during their maintenance by transducing a substantially homogeneous cell composition with a recombinant DNA construct. Preferably, a retroviral vector is employed for the introduction of the DNA construct into the cell. The resulting cells can then be grown under conditions similar to those for unmodified cells, whereby the modified cells can be expanded and used for a variety of purposes.

For genetic modification of the cells, usually a retroviral vector will be employed, however any other suitable viral vector or delivery system can be used. Combinations of retroviruses and an appropriate packaging line are also suitable, where the capsid proteins will be functional for infecting human cells. Various amphotropic virus-producing cell lines are known, including, but not limited to, PA12 (Miller et al. (1985) Mol. Cell. Biol. 5:431-437); PA317 (Miller et al. (1986) Mol. Cell. Biol. 6:2895-2902); and CRIP. Danos et al. (1988) Proc. Natl. Acad. Sci. USA 85:6460-6464. Non-amphotropic particles are suitable too, e.g., particles pseudotyped with VSVG, RD114 or GALV envelope and any other known in the art.

Possible methods of transduction include direct co-culture of the cells with producer cells, e.g., by the method of Bregni et al. (1992) Blood 80:1418-1422, or culturing with viral supernatant alone or concentrated vector stocks with or without appropriate growth factors and polycations, e.g., by the method of Xu et al. (1994) Exp. Hemat. 22:223-230; and Hughes et al. (1992) J. Clin. Invest. 89:1817.

Gene transfer technology, based on retrovirus-mediated transduction, can be used to genetically modify the CTLs activated by the AAPC. Such genetic modification can be for the purpose of expressing therein molecules with therapeutic relevance, e.g., markers, suicide genes or molecules with anti-apoptotic or costimulatory functions.

Upon reintroduction of the genetically modified cells into the host and subsequent differentiation, T cells are induced that are specifically directed against the specific antigen. “Induction” of T cells can include inactivation of antigen-specific T cells such as by deletion or anergy. Inactivation is particularly useful to establish or reestablish tolerance such as in organ transplantation and autoimmune disorders respectively. The modified DCs can be administered by any method known in the art including, but not limited to, subcutaneous, intranodal and directly to the thymus.

The modified cells can be administered in any physiologically acceptable vehicle, normally intravascularly, although they may also be introduced into bone or other convenient site where the cells may find an appropriate site for regeneration and differentiation (e.g., thymus). Usually, at least 1×10⁵ cells will be administered, preferably 1×10⁶, eventually reaching 1×10¹⁰, or more. The cells can be introduced by injection, catheter, or the like. If desired, factors can also be included, including, but not limited to, interleukins, e.g. IL-2, IL-3, IL-6, and IL-11, as well as the other interleukins, the colony stimulating factors, such as G-, M- and GM-CSF, interferons, e.g. γ-interferon and erythropoietin.

The term “polypeptide,” “peptide” and “protein” are used interchangeably herein to refer to polymers of amino acid residues of any length. The polymer can be linear or branched, it can comprise modified amino acids or amino acid analogs, and it can be interrupted by chemical moieties other than amino acids. The terms also encompass an amino acid polymer that has been modified naturally or by intervention; including, but not limited to, disulfide bond formation, glycosylation, lipidation, acetylation, phosphorylation, or any other manipulation or modification, such as conjugation with a labeling or bioactive component. Unless stated or implied otherwise, the term antigen-binding fragment includes any polypeptide monomer or polymer with immunologic specificity, including the intact antibody, and smaller and larger functionally equivalent polypeptides, as described herein.

A “fusion polypeptide” is a polypeptide comprising contiguous peptide regions in a different position than would be found in nature. The regions can normally exist in separate proteins and are brought together in the fusion polypeptide; they can normally exist in the same protein but are placed in a new arrangement in the fusion polypeptide; or they can be synthetically arranged. For instance, as described below, the invention encompasses recombinant proteins (and the polynucleotides encoding the proteins or complementary thereto) that are comprised of a functional portion of an antigen-binding fragment and a toxin. Methods of making these fusion proteins are known in the art and are described for instance in WO93/07286.

A “functionally equivalent fragment” of a polypeptide varies from the native sequence by any combination of additions, deletions, or substitutions while preserving at least one functional property of the fragment relevant to the context in which it is used.

A “signal peptide” or “leader sequence” is a short amino acid sequence that directs a newly synthesized protein through a cellular membrane, usually the endoplasmic reticulum (ER) in eukaryotic cells, and either the inner membrane or both inner and outer membranes of bacteria. Signal peptides are typically at the N-terminus of a polypeptide and are removed enzymatically between biosynthesis and secretion of the polypeptide from the cell or through the membrane of the ER. Thus, the signal peptide is not present in the secreted protein but is present only during protein production.

Substitutions can range from changing or modifying one or more amino acid to complete redesign of a region, such as the V region. Amino acid substitutions, if present, are preferably conservative substitutions that do not deleteriously affect folding or functional properties of the peptide. Groups of functionally related amino acids within which conservative substitutions can be made are glycine/alanine; valine/isoleucine/leucine; asparagine/glutamine; aspartic acid/glutamic acid; serine/threonine/methionine; lysine/arginine; and phenylalanine/tryosine/tryptophan. Antigen-binding fragments can be glycosylated or unglycosylated, can be modified post-translationally (e.g., acetylation, and phosphorylation) or can be modified synthetically (e.g., the attachment of a labeling group).

Recombinant methods are well known in the art. The practice of the invention employs, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (Gait, ed., 1984); “Animal Cell Culture” (Freshney, ed., 1987); “Methods in Enzymology” (Academic Press, Inc.); “Handbook of Experimental Immunology” (Wei & Blackwell, eds.); “Gene Transfer Vectors for Mammalian Cells” (Miller & Calos, eds., 1987); “Current Protocols in Molecular Biology” (Ausubel et al., eds., 1987); “PCR: The Polymerase Chain Reaction”, (Mullis et al., eds., 1994); and “Current Protocols in Immunology” (Coligan et al., eds., 1991). These techniques are applicable to the production of the polynucleotides and polypeptides, and, as such, can be considered in making and practicing the invention. Particularly useful techniques for are discussed in the sections that follow.

The polynucleotides of the invention can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as can be desirable to provide embodiments of this invention.

Methods of Treatment

Also included in this invention are methods for treating a variety of disorders as described herein and/or known in the art. The methods comprise administering an amount of a pharmaceutical composition containing a composition of the invention in an amount effective to achieve the desired effect, be it palliation of an existing condition or prevention of recurrence. For treatment of cancer, the amount of a pharmaceutical composition administered is an amount effective in producing the desired effect. An effective amount can be provided in one or a series of administrations. An effective amount can be provided in a bolus or by continuous perfusion. Suitable active agents include the anti-neoplastic drugs and bioresponse modifiers described above and effector cells such as those described by Douillard et al. (1986) Hybridomas (Supp. 1:5139).

Pharmaceutical compositions and treatment modalities are suitable for treating a patient by either directly or indirectly eliciting an immune response against neoplasia. An “individual,” “patient” or “subject” is a vertebrate, preferably a mammal, more preferably a human. Mammals include, but are not limited to: humans, wild animals, feral animals, farm animals, sport animals, and pets. A “cancer subject” is a mammal, preferably a human, diagnosed as having a malignancy or neoplasia or at risk thereof.

As used herein, “treatment” refers to clinical intervention in an attempt to alter the disease course of the individual or cell being treated, and can be performed either for prophylaxis or during the course of clinical pathology. Therapeutic effects of treatment include without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis.

The “pathology” associated with a disease condition is any condition that compromises well-being, normal physiology, or quality of life. This can involve, but is not limited to, destructive invasion of affected tissues into previously unaffected areas, growth at the expense of normal tissue function, irregular or suppressed biological activity, aggravation or suppression of an inflammatory or immunologic response, increased susceptibility to other pathogenic organisms or agents, and undesirable clinical symptoms such as pain, fever, nausea, fatigue, mood alterations, and such other disease-related features as determined by an attending physician.

An “effective amount” is an amount sufficient to effect a beneficial or desired clinical result upon treatment. An effective amount can be administered to a patient in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition and the form and effective concentration of the antigen-binding fragment administered.

For adoptive immunotherapy using antigen-specific T cells, cell doses in the range of 10⁹ are typically infused. Rosenberg (1991); Melief and Kast (1995); Riddell and Greenberg (1995); Rooney et al. (1998); and O'Reilly et al. (1998). Based on a conservative estimation of 8-fold expansion obtained with AAPC^{A2G} or AAPC^{A2M} after two stimulations (Table 1), generation of 10⁹ CD8⁺ T cells would require about 1.2×10⁸ peripheral blood CD8⁺ T cells as the starting material, thus requiring 25>500 ml of blood. If additional cells were needed or if the starting cell number was less, a third round of stimulation or further nonspecific activation using, for example, beads coated with anti-CD3 and anti-CD28 antibodies could be envisaged. Levine et al. (1997) J. Immunol. 159:5921-5930.

Suitable human subjects for cancer therapy further comprise two treatment groups, which can be distinguished by clinical criteria. Patients with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in this invention is administered to these patients to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of cancer, but have been responsive to another mode of therapy. The prior therapy can have included (but is not restricted to, surgical resection, radiotherapy, and traditional chemotherapy. As a result, these individuals have no clinically measurable tumor. However, they are suspected of being at risk for progression of the disease, either near the original tumor site, or by metastases.

“Adjuvant” as used herein has several meanings, all of which will be clear depending on the context in which the term is used. In the context of a pharmaceutical preparation, an adjuvant is a chemical or biological agent given in combination (whether simultaneously or otherwise) with, or recombinantly fused to, an antigen to enhance immunogenicity of the antigen. For review see, Singh et al. (1999) Nature Biotech. 17:1075-1081. Isolated DCs have also been suggested for use as adjuvants. Compositions for use therein are included in this invention. In the context of cancer diagnosis or treatment, adjuvant refers to a class of cancer patients with no clinically detectable tumor mass, but who are suspected of being at risk of recurrence.

This group can be further subdivided into high-risk and low-risk individuals. The subdivision is made on the basis of features observed before or after the initial treatment. These features are known in the clinical arts, and are suitably defined for each different cancer. Features typical of high-risk subgroups are those in which the tumor has invaded neighboring tissues, or who show involvement of lymph nodes.

Another group have a genetic predisposition to cancer but have not yet evidenced clinical signs of cancer. For instance, women testing positive for a genetic mutation associated with breast cancer, but still of childbearing age, can wish to receive one or more of the antigen-binding fragments described herein in treatment prophylactically to prevent the occurrence of cancer until it is suitable to perform preventive surgery.

Human cancer patients, including, but not limited to, glioblastoma, melanoma, neuroblastoma, adenocarcinoma, glioma, soft tissue sarcoma, and various carcinomas (including small cell lung cancer) are especially appropriate subjects. Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas.

The patients can have an advanced form of disease, in which case the treatment objective can include mitigation or reversal of disease progression, and/or amelioration of side effects. The patients can have a history of the condition, for which they have already been treated, in which case the therapeutic objective will typically include a decrease or delay in the risk of recurrence.

The invention will be further described by way of the following examples provided to illustrate but not limit the invention.

### EXAMPLE 1

### Vector Construction

cDNAs were cloned into the NcoI and BamHI sites of the SFG vector Backbone. Riviere et al. (1995) Proc. Natl. Acad. Sci. USA 92:6733-6737. A dicistronic vector encoding neomycin phosphotransferase 3′ of the encephalomyocarditis virus internal ribosomal entry site (Gallardo et al. (1997) Gene Ther. 4:1115-1119) was constructed to express HLA A2.1 (kind gift of Drs. S. Y. Young and N. Cereb). A dicistronic vector encoding puromycin-N-acetyltransferase was used for the minigenes encoding the different peptides used in this study. The human CD8 leader was fused to the peptide antigens to target the endoplasmic reticulum. Monocistronic vectors were constructed for the human β2-microglobulin (kind gift of Dr. S. Y. Young), CD80 (Gong et al. (1999)), CD54, and CD58 (kind gift of Dr. M. Dustin).

### EXAMPLE 2

### Gene Transfer Procedures

293GPG packaging cells (Ory et al. (1996) Proc. Natl. Acad. Sci. USA 93:11400-11406) were transfected with each plasmid by CaCl₂ as described in Riviere and Sadelain, in, Gene therapy protocols (ed. Robbins) pp. 59-78 (Humana Press, Totowa, N.J., (1997). A total of 5×10⁴ NIH 3T3 cells (ATCC) were plated in a 6 cm plate and cultured in Dulbecco's modified Eagle medium (DMEM; Mediatech, Hermdon, Va.) with 10% heat-inactivated donor calf serum (DCS; Hyclone, Logan, UT), penicillin at 100 U ml⁻¹, and streptomycin at 100 μg ml⁻¹. They were infected the day after with cell-free retroviral supernatant (0.45 μm filtration, Acrodisc; Pall Corporation, Ann Arbor, Mich.) in the presence of polybrene (Sigma, St. Louis, Mo.) at 8 μg ml⁻¹for 16 h.

Geneticin (Sigma) was added at 1.2 mg ml⁻¹ to the medium for two weeks to select the cells expressing A2.1. Puromycin (Sigma) was added at 3 μg ml⁻¹ to the medium for one week to select cells expressing the vector-encoded peptide. After transduction with a monocistronic vector, if gene transfer was extremely efficient (>95%), no cell purification was required. If gene transfer was less efficient, transduced cells were purified by using magnetic beads (Dynal, Oslo, Norway) or flow cytometry (Becton Dickinson, San Jose, Calif.).

### EXAMPLE 3

### Generation of DCs and T Cell Purification

Peripheral blood was obtained from normal HLA A2.1⁺ donors in heparinized tubes. HLA typing was performed by PCR in the HLA laboratory at MSKCC. Peripheral blood mononuclear cells (PBMC) were isolated by density gradient centrifugation on lymphocyte separation medium (Accurate Chemical & Scientific Corporation, Westbury, N.Y.). Dendritic cells were generated as described. Bender et al. (1996) J. Immunol. Met. 196:121-135; and Romani et al. (1996).

Briefly, the T cell-depleted (ER⁻) population was prepared by rosetting with sheep red blood cells (Colorado Serum Company, Denver, Co.). O'Doherty et al. (1993). Two million ER⁻ cells were plated per well in six-well plates. GM-CSF (Immunex, Seattle, Wash.) and IL-4 (R&D Systems, Minneapolis, Minn.) were added at 1,000 U ml⁻¹ every second day for eight days. Conditioned medium (CM) was prepared by adding 50×10⁶ ER⁻ cells on Petri dishes coated with human γ-globulins (Sigma) at 10 mg ml^{-1.}

Nonadherent cells were removed and the CM, collected after 24 h, was added (a half or a third of the final volume) to the cells for four days to get fully mature DCs. After four days with CM, the cells had a phenotype of fully mature DCs: they had lost the expression of CD14, expressed high levels of CD40, CD80, MHC class I and class II molecules, and had acquired the expression of the specific marker CD83. T cells were purified as described. Bhardwaj et al. (1994) J. Clin. Invest. 94:797-807. Briefly, the T cell-enriched (ER⁺) population was collected from the same donors. After lysis of the sheep red blood cells and three washes in phosphate-buffered saline (PBS) with 2% heat-inactivated fetal calf serum (FCS, Hyclone), B cells, natural killer cells, monocytes-macrophages, and activated T cells were depleted. This was accomplished by incubating cells with mouse IgG mAbs directed against CD11b, CD16, and HLA DP, DQ, DR (Pharmingen, San Diego, Calif.) at 1 μg per million cells for 30 min, followed by a panning on Petri dishes coated with goat anti-mouse IgG (Caltag, Burlingame, Calif.) as described by Young et al. (1990) J. Exp. Med. 171:1315-1332. After three washes in PBS with 2% FCS, the T cells were resuspended at a final concentration of 10 million cells/ml. Dendritic cells were maintained in RPMI 1640 (Mediatech) with 10% FCS. T lymphocytes were maintained in AIM V medium (Life Technologies, Rockville, Md.) without serum. Penicillin at 100 U ml⁻¹ and streptomycin at 100 μg ml⁻¹ were added to all the cultures.

### EXAMPLE 4

### Flow Cytometry Analysis

To analyze the phenotype of the AAPCs, we used antibodies against human β2-microglobulin, A2.1 (kind gifts of Dr. S. Y. Young), B7.1 (Pharmingen), ICAM-1, and LFA-3 (Becton Dickinson). Anti-CD14, CD80, CD40, HLA DR (Becton Dickinson), and anti-CD83 (Immunex, Marseilles, France) antibodies were used to evaluate the level of maturation of the DCs. To verify the purity of the preparations of T cells and to study the phenotype of these T cells, we stained cells with antibodies anti-CD19, CD14, CD56, CD16, CD3, CD4, CD8, CD25, CD69, and HLA DR (Becton Dickinson).

### EXAMPLE 5

### Stimulation of Specific CTLs

DCs were pulsed with the peptide (10 M) for 2 h at room temperature in RPMI without serum. Coculture with T cells was established at the ratio 10 T lymphocytes to 1 DC in 24-well plates, with 1 million T cells per well for 8-10 days, in RPMI with 10% FCS. Artificial APCs were irradiated (1,500 Gy) and plated the day before in 24-well plates at the concentration 105 cells/ml in AIM V medium with 5% DCS, 500 μl per well. T cells were resuspended in AIM V medium at 2×10⁶ cells/ml, added to AAPCs at 500 l per well, and cultured for 8-10 days. IL-2 (Chiron, St. Louis, Mo.) was added to the cultures after seven days (20 IU ml⁻, every third day). To restimulate the T cells 10-14 days after induction, they were cocultured with AAPCs following the same procedure, with 10⁵ T cells per well for 10-14 days. Every third day, IL-2 at 20 IU ml⁻¹ was added.

### EXAMPLE 6

### Cytotoxicity Assays

Standard chromium release assays were performed, using as target cells. Transfer associated with antigen processing (TAP) protein-deficient HLA A2.1⁺ T2 cells (kind gift of Dr. J. W. Young), loaded with the different peptides (10 μM, 1 h at room temperature, in RPMI without serum) before pulsing with ⁵¹Cr for 1 h at 37° C. We used 5,000 T2 cells per well in 96 V-bottom plates at different effector:target cell (E:T) ratios for 4 h. We also used SK-MEL23 and SK-MEL28 cells as targets (kind gifts of Dr. P. Chapman). They are, respectively, HLA A2.1⁺ and HLA A2.1⁻ melanoma cell lines that express MART-1 and gp 100 antigens. Chen et al. (1996) Proc. Natl. Acad. Sci. USA 93:5915-5919. SK-MEL cells were pulsed with ⁵¹Cr as for the T2 cells. We performed 16 h cytotoxicity assays with 1,000 target cells per well. Specific ⁵¹Cr release was calculated using the formula ((⁵¹ Cr release—spontaneous release)/(maximum release—spontaneous release)) 100. Lytic units (LU) were calculated according to equation (12) in Bryant et al. (1992) J. Immunol. Met. 146:91-103.

### EXAMPLE 7

### Peptide Synthesis

All the peptides were synthesized in the Peptide Synthesis Facility at MSKCC, resuspended in 50% (vol/vol) RPMI-dimethylsulfoxide (Sigma), and stored at ⁻20° C. The following peptides were used in this study: the influenza matrix protein-derived peptide₅₈₋₆₆ GILGFVFTL (flu peptide (SEQ ID NO: 43)); the MART-1 protein-derived peptide₂₇₋₃₅ AAGIGILTV (SEQ ID NO: 44); the gp 100-modified peptide₂₀₉₋₂₁₇ IMDQVPFSV ((SEQ ID NO: 45) g209-2M, which efficiently induces CTLs against the natural gp 100 peptide). Parkhurst et al. (1996).

### EXAMPLE 8

### Construction of AAPCs

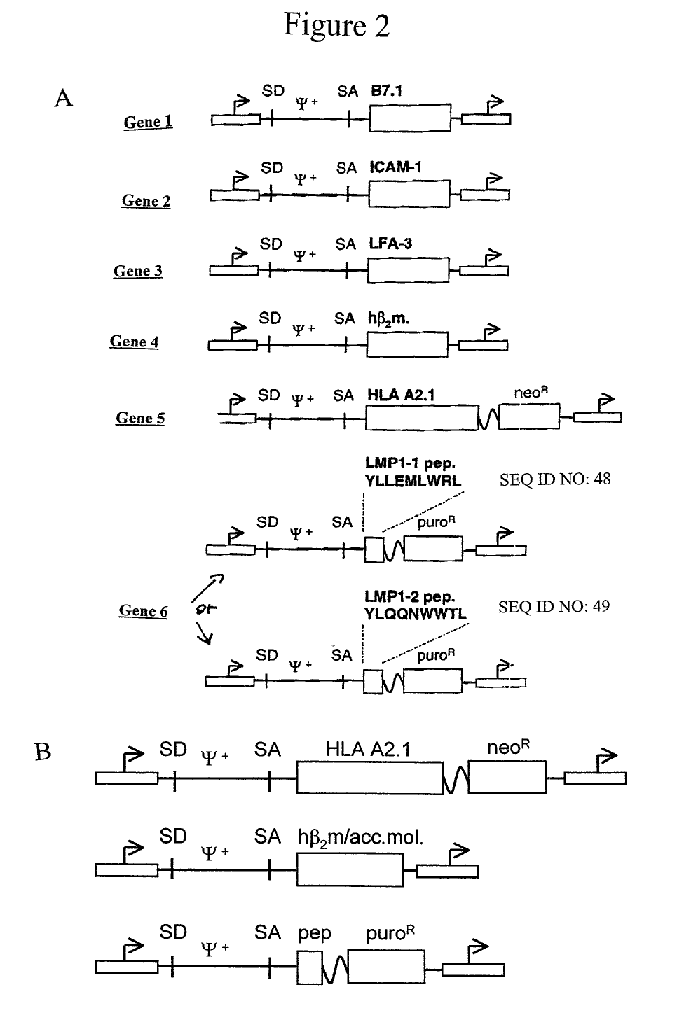

To generate AAPCs restricted to the HLA class I A2.1 molecule (AAPCA2), replication-incompetent retroviral vectors were used to sequentially transduce NIH/3T3 fibroblasts with five vectors encoding, respectively, human B7.1, ICAM-1, LFA-3, human β2-microglobulin, and HLA A2.1 (FIGS. 2A and 2B). To maximize and sustain expression of a specific HLA-peptide complex, a dicistronic vector encoding an HLA-restricted epitope and puromycin-N-acetyltransferase was used (FIG. 2A).

The expression of the peptide, targeted to the endoplasmic reticulum by the human CD8 leader, was maintained under selective pressure with puromycin. High-level and stable expression of the different transmembrane molecules was obtained (FIG. 2C). By flow cytometry analysis, the levels of expression of A2.1, B7.1, ICAM-1, and LFA-3 were comparable to those measured on mature A2.1⁺ dendritic cells. In FIG. 2, (A) Monocistronic retroviral vectors expressed human β2-microglobulin (hβ2m) and the accessory molecules (acc. mol.) CD80, CD54, and CD58 (top). Dicistronic vectors were generated for HLA A2.1 and the peptide coding sequence (pep), respectively linked by an internal ribosomal entry site to neomycin phosphotransferase (neo^{R}, middle) or puromycin-N-acetyltransferase (puro^{R}, bottom). SD, Splice donor site; SA, splice acceptor site; ψ⁺, extended packaging signal. (C) Flow cytometry analysis of HLA A2.1, CD80, CD54, and CD58 expression in AAPCs. The same cells are stained for each molecule as indicated. Solid lines correspond to transduced NIH 3T3 cells and dashed lines to untransduced cells. For HLA A2.1, the dotted line corresponds to cells transduced with HLA A2.1 without human β2-microglobulin, and the solid line to cells transduced with both cDNAs.

### EXAMPLE 9

### Artificial APCs Efficiently Stimulate Flu-specific Cytotoxic T cell Responses

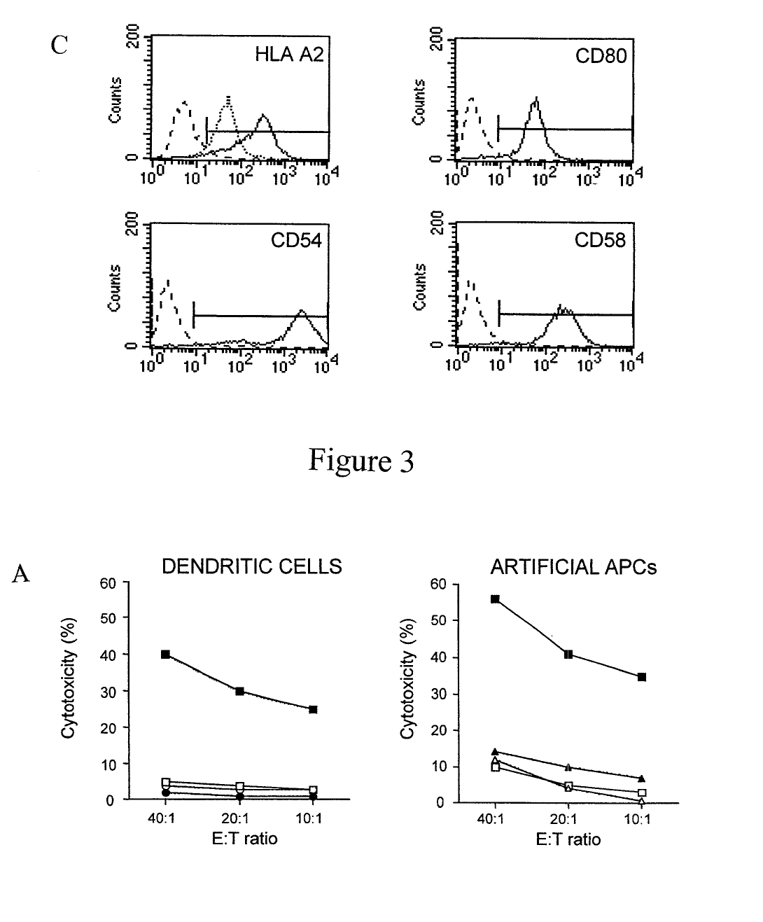

Peripheral blood T lymphocytes harvested from HLA A2.1⁺ donors were stimulated either with primary autologous DCs pulsed with the flu peptide or AAPC^{A2} genetically engineered to express the same peptide (AAPC^{A2F}). Highly purified populations of T cells were prepared by positive selection (sheep red blood cells rosetting) and depletion of monocytes-macrophages, B cells, natural killer cells, and activated T cells. After 8-10 days of stimulation, T lymphocytes cultured with AAPC^{A2F} exhibited strong flu-specific cytolytic activity (FIG. 3A). Typically the cytolytic activity was 1.6- to 4-fold higher than that obtained with primary dendritic cells pulsed with the flu peptide (115 and 65 lytic units, respectively, in FIG. 3A). The background on unpulsed target cells or on target cells pulsed with an irrelevant peptide was always lower than 5% (FIG. 3A). In FIG. 3, (A) Cytotoxicity of T cells from HLA A2.1⁺ donor stimulated with primary autologous dendritic cells (left panel) or AAPC^{A2F} (right panel). Standard ⁵¹Cr release assays were performed using TAP-deficient A2.1⁺ T2 target cells pulsed with the flu peptide (filled symbols) or the irrelevant MART-1 peptide (open symbols). Squares correspond to T cells stimulated against the flu peptide; circles to T cells stimulated without the relevant peptide. Y-axis, percentage of specific ⁵¹Cr release; X-axis, effector:target (E:T) ratios. (B) Flow cytometry analysis of CD8⁺ T cells before (upper panels) and after (lower panels) cocultivation with HLA A2.1⁺ AAPCs encoding the flu peptide. T cells were stained with a fluorescein isothiocyanate (FITC)-labeled antibody against CD8 (X-axis) and, from left to right, phycoerythrin-labeled antibodies against CD25, CD69, and DR (Y-axis). Results are from one of six experiments with one representative donor.

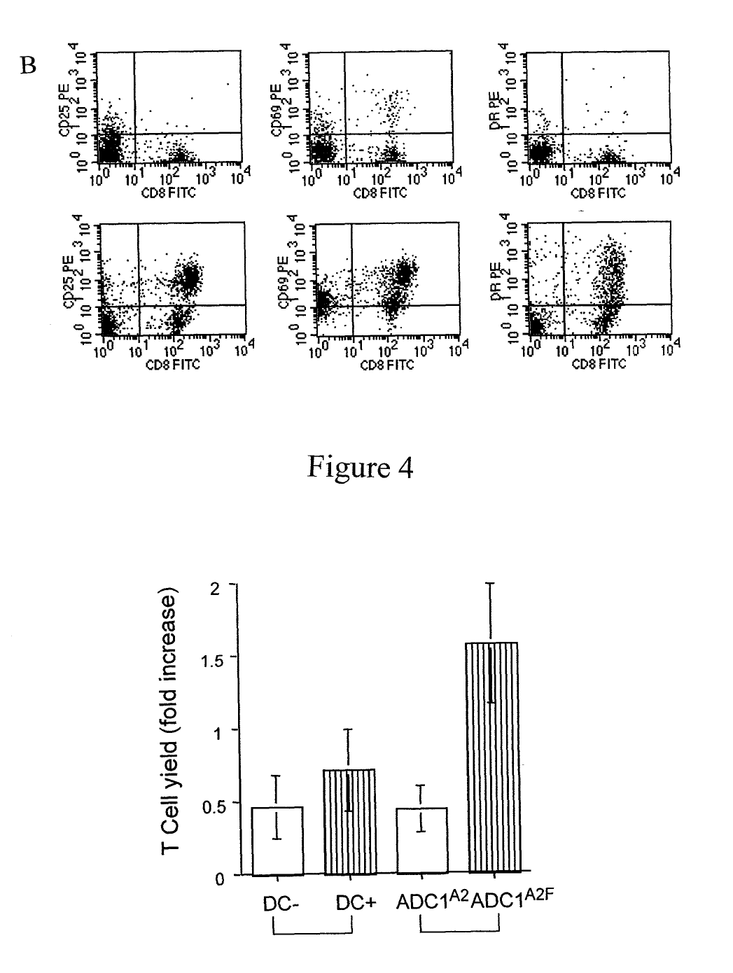

Examination of the cell surface phenotype of the CD8⁺ cells showed a strongly activated profile, as reflected by the high level of expression of CD25 (low-affinity interleukin-2 receptor), CD69 (very early activation marker), and HLA DR (FIG. 3B). Fewer than 5% of the purified T cells expressed these markers at the start of the coculture (FIG. 3B). Furthermore, absolute cell counts of CD8⁺ T cells on days 8-10 showed a higher cell yield following coculture with AAPCs than with primary DCs, about 2-fold higher in six different experiments (P<0.001, FIG. 4). Such an expansion of CD8⁺ T cells could not be reached with AAPCs expressing ICAM-1 and/or LFA-3 in the absence of B7.1. The presence of both accessory molecules increased the effect of B7.1 by a factor of 2. In FIG. 4, CD8 T cell yield (fold increase, mean +/−s.d) is indicated on the y-axis, corresponding to six independent experiments with the same donor. The yield was significantly greater with AAPC^{A2F} than with flu peptide-pulsed DCs (P<0.001, Student's t-test). Similar results were obtained with two other donors. Open bars, stimulation without relevant peptide; hatched bars, stimulation against flu peptide.

### EXAMPLE 10

### Artificial APCs Efficiently Induce CTLs Specific for Self-antigens

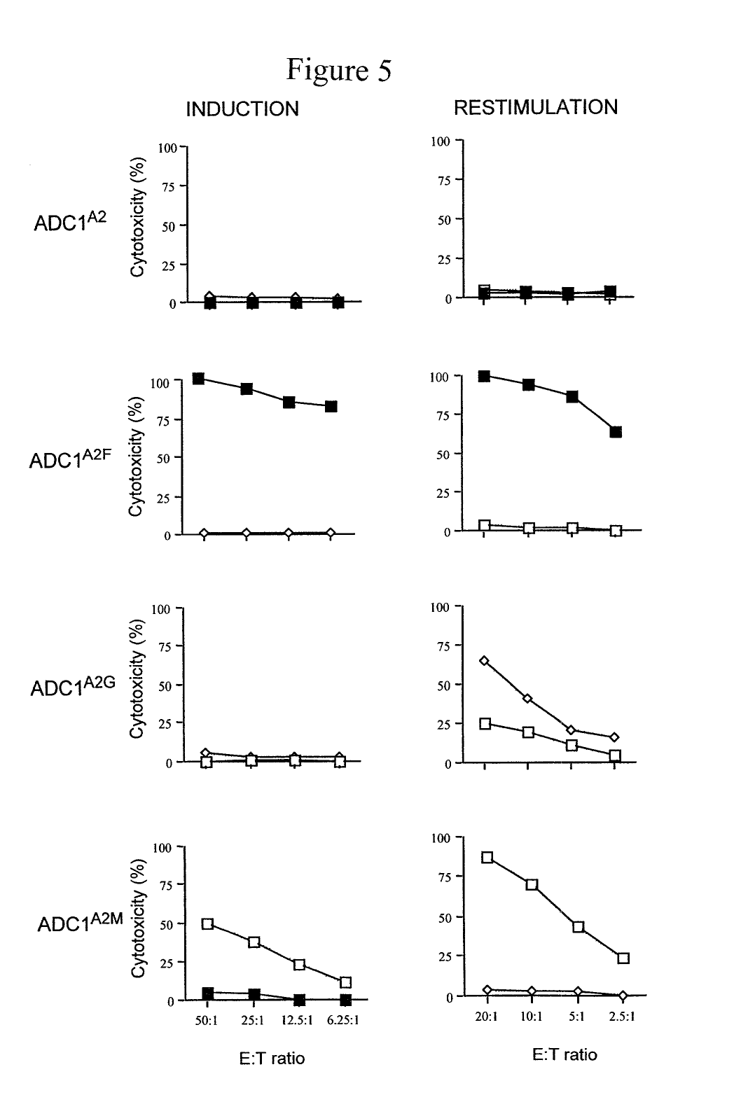

To address whether AAPCs could induce a response against self-antigens, HLA A2.1⁺ AAPCs encoding two peptides expressed in human HLA A2.1⁺ melanoma cells were generated. One peptide is derived from the MART-1 protein and the other from the gp100 protein including an amino acid substitution to enhance binding to HLA A2.1. Kawakami et al. (1994); and Parkhurst et al. (1996). Highly purified T cells harvested from three HLA A2.1⁺ donors were cultured with AAPCs expressing the MART-1 (AAPC^{A2M}) or gp 100 (AAPC^{A2G}) derived peptide, using AAPC^{A2F} as control. After the first stimulation, as expected, a high response was obtained against the flu peptide in all three donors. In one donor, we readily detected a measurable CTL response against the MART-1 peptide (FIG. 5).

After restimulation with the respective AAPCs, a readily detectable cytolytic response was obtained against all three peptides while the flu response further increased (FIG. 5). In FIG. 5, Cytotoxicity was measured after the first stimulation (left panels) or after restimulation with the same AAPCs (right panels). Four HLA A2.1⁺ AAPCs were used: AAPC^{A2} without peptide (AAPC A2), AAPCA2 expressing the flu peptide (AAPC^{A2F}), the gp100-derived peptide (AAPC^{A2G}), or the MART-1-derived peptide (AAPCA2M). Cytotoxicity assays were performed with T2 cells as targets. Filled symbols correspond to target cells pulsed with the relevant peptide; open symbols to target cells pulsed with an irrelevant peptide (MART-1 peptide for CTLs stimulated with AAPC^{A2F}, flu peptide for CTLs stimulated with AAPCA2, AAPC^{A2G} or Y-axis, percentage of specific ⁵¹Cr release; X-axis, effector:target (E:T) ratios.

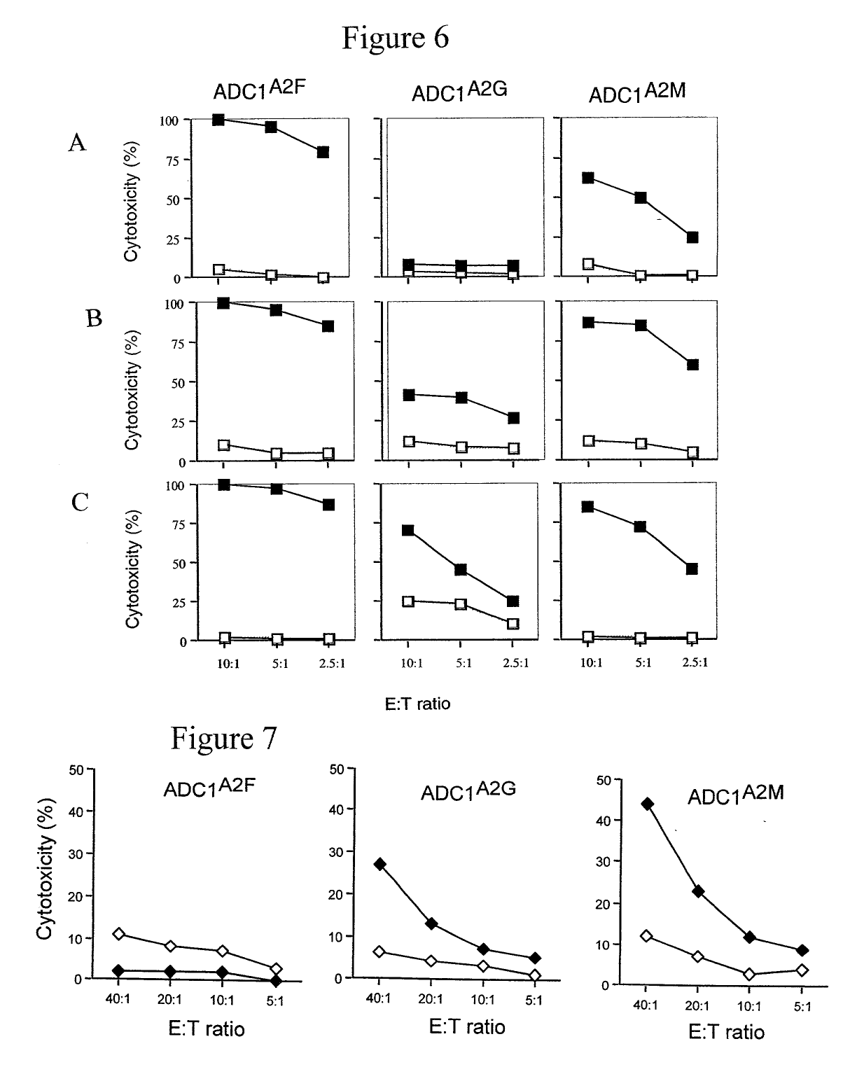

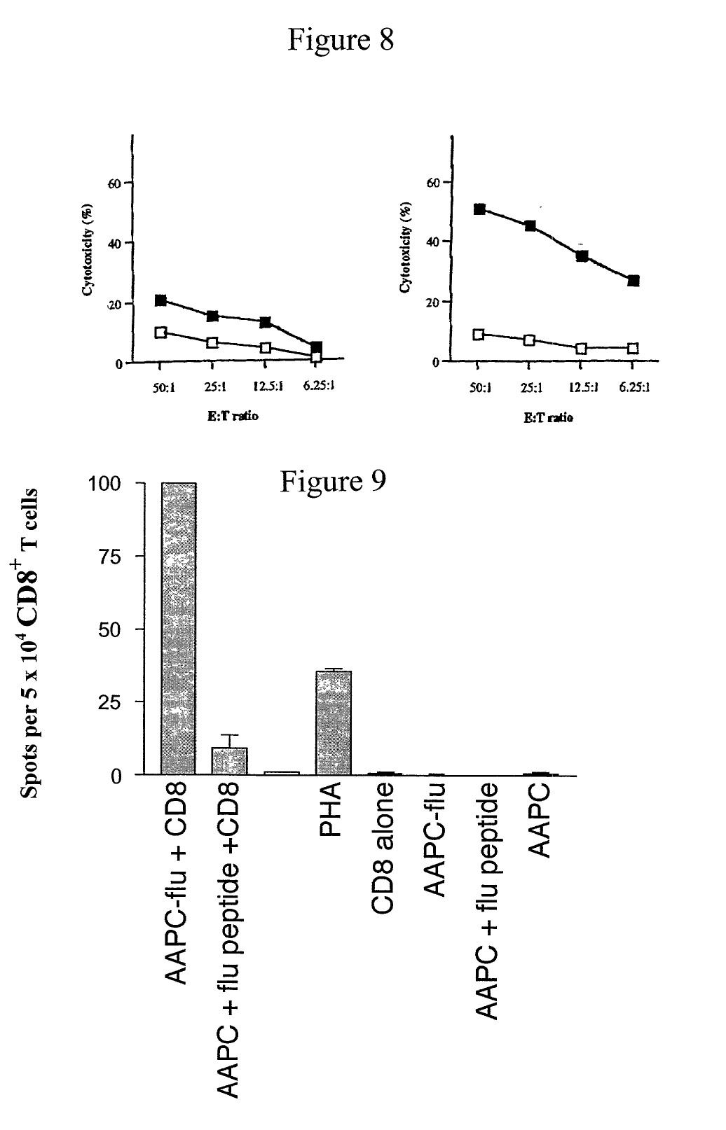

After restimulation, the response against the MART-1 peptide was of comparable magnitude to that obtained against the flu peptide after the first stimulation. The cytolysis obtained after two stimulations for the three peptides in three HLA A2.1⁺ donors is shown in FIG. 6. In FIG. 6, T cells purified from three HLA A2.1⁺ donors (A, B, C) were stimulated twice by AAPC^{A2F} AAPC^{A2G} or AAPC^{A2M}. Cytotoxicity stimulation was performed on T2 cells as described in FIGS. 3 and 5. Y-axis, percentage of specific ⁵¹Cr release; X-axis, effector:target (E:T) ratios. FIG. 8 illustrates the cytotoxicity results with EBV/LMP 1.1 peptide.

All three donors showed strong responses against the MART-1 peptide, and two out of three significantly responded to the gp 100 peptide. Results obtained with these three donors in terms of cellular expansion and antigen specificity for all three peptides investigated in this study are summarized in Table 2. In Table 2, the results obtained with expansion and antigen specificity of CD8⁺ T cells after one or two AAPC stimulation are shown. Between 6 and 18 million HLA A2.1⁺ donor T cells were plated on the different AAPCs on day 0. Cells were counted and stained for CD8, CD25, CD69, and HLA DR expression after the first (A) and second (B) stimulations. T cell numbers correspond to a starting number of 1×10⁶ CD8⁺ T cells. Specific cytotoxicity measured against T2 cells pulsed with the immunizing peptide (as in FIGS. 5 and 6) is shown as the 10:1 E:T ratio. Background activity measured at the same ratio against an irrelevant peptide (as in FIGS. 5 and 6) was subtracted.

TABLE 2

| | | ADC1^{A2} | | ADC1^{A2F} | | ADC1^{A2G} | | ADC1^{A2M} | |
|---|---|---|---|---|---|---|---|---|---|
| | | 1^{st} | 2^{nd} | 1^{st} | 2^{nd} | 1^{st} | 2^{nd} | 1^{st} | 2^{nd} |
| Donor | | stim | stim | stim | stim | stim | stim | stim | stim |
| 1 | Abs nb of | 0.2 | 0 | 1.5 | 9 | 0.34 | 2.8 | 0.36 | 3 |
| | cells(input = 1M) | | | | | | | | |
| | Specific | 10 | 5 | 75 | 100 | 1 | 5 | 20 | 70 |
| | cytotoxicity(%) | | | | | | | | |
| 2 | Abs nb of | 1 | 5.8 | 3.3 | 33 | 0.95 | 12.5 | 0.8 | 14.6 |
| | cells(input = 1M) | | | | | | | | |
| | Specific | 15 | 5 | 100 | 100 | 5 | 50 | 10 | 80 |
| | cytotoxicity(%) | | | | | | | | |
| 3 | Abs nb of | 0.46 | 0.2 | 2 | 13.2 | 0.93 | 3.6 | 0.72 | 3.1 |
| | cells(input = 1M) | | | | | | | | |
| | Specific | 0.5 | 0.5 | 100 | 100 | 5 | 45 | 45 | 80 |
| | cytotoxicity(%) | | | | | | | | |

After two rounds of stimulation with AAPC^{A2F} , CD8⁺ T cell yields increased 25-to 80-fold. After two rounds of stimulation with AAPC^{A2G} or AAPC^{A2M} , CD8⁺ T cell yields increased 8- to 30-fold. CD8⁺ T cells were highly activated, as indicated by their elevated expression of CD25, CD69, and HLA DR (with phenotypic profiles similar to those in FIG. 3B).

Cytotoxic T lymphocytes induced by AAPC^{A2} that encode the MART-1 or gp 100-derived peptide specifically lyse HLA A2.1⁺ melanoma cells. To address whether T cells induced by AAPCs recognize and lyse melanoma cells in an HLA-restricted manner, cytotoxicity assays were performed using HLA A2.1⁺ and HLA A2.1⁻ melanoma cells as targets. The SK-MEL23 and SK-MEL28 cell lines both express MART-1 and gp 100 proteins and are, respectively, A2.1⁺ and A2.1-. Chen et al. (1996). T cells induced by AAPC^{A2G} or AAPC^{A2M} effectively lysed SK-MEL23 cells, showing, respectively, 30 and 45% lysis at the 40:1 effector:target ratio (FIG. 7). These T cells were HLA restricted as they failed to lyse SK-MEL28. On the other hand, T cells stimulated by AAPC^{A2F} failed to lyse SK-MEL23, demonstrating their high specificity. The low-level cytoxicity against SK-MEL28 was comparable whether the T cells had been previously stimulated by AAPC^{A2F} , AAPCA23, or AAPC^{A2M} (FIG. 7). In FIG. 7, cytotoxicity of T cells of donor C (FIG. 6) induced by AAPC^{A2F} , AAPC^{A2G} , or AAPC^{A2M} against SK-MEL23 (HLA A2.1⁺, filled symbol) and SK-MEL28 (HLA A2.1-, open symbol). Y-axis, percentage of specific ⁵¹Cr release; X-axis, effector:target (E:T) ratios. Cytotoxic T lymphocytes induced by AAPC^{A2M} and AAPC^{A2G} efficiently lysed SK-MEL23. The same low level of cytotoxicity was obtained against SK-MEL28 whether the CTLs were activated on AAPC^{A2F} , AAPC^{A2M}, or AAPC^{A2G}. Similar results were obtained with donor B of FIG. 6.

Xenogeneic fibroblasts expressing retrovirally transduced HLA class I-peptide complexes along with CD80, CD54, and CD58 efficiently stimulate peripheral blood T cells of donors sharing the same HLA molecule. The AAPCs express a human tripartite complex comprising one HLA molecule, human β2-microglobulin, and one encoded peptide. The total yield of CD8⁺ T cells obtained by stimulation with AAPCs is higher than that achieved with peptide-pulsed autologous dendritic cells, albeit under distinct culture conditions. Several factors may contribute to the high efficiency of the AAPCs. The level of cell surface expression of HLA A2.1, CD80, CD54, and CD58 is elevated, comparable to that of mature primary HLA A2.1⁺ DCs.

The density of the specific HLA-peptide complex may also play an important role. Artificial APCs endogenously express under selective pressure the relevant peptide, which is targeted to the endoplasmic reticulum where peptides are loaded onto nascent HLA class I complexes. Anderson et al. (1991) J. Exp. Med. 174:489-492; and Lehner and Cresswell (1996) Curr. Opin. Immunol. 8:59-67. Expression of the specific complex is therefore maintained irrespectively of the turnover of these complexes at the cell membrane, which is not the case with peptide-pulsed APCs, including artificial APCs derived from Drosophila cells. Sprent et al. (1997) Adv. Exp. Med. Biol. 417:249-254. Another advantage of using mouse fibroblasts compared to Drosophila cells is their stability in culture and ease of manipulation. Another important difference is the ability of animal cells such as fibroblasts to process and present antigen in a therapeutically effective manner. Improperly processed or unprocessed antigens will not be recognized by T cells. The low ability of fibroblasts to process and load peptides onto MHC molecules, as compared to professional APCs, may also contribute to enhanced expression of the specific HLA-peptide complex by decreasing simultaneous presentation of irrelevant peptides. Sprent (1995) Curr. Biol. 5:1095-1097; and Mellman et al. (1998) Trends Cell Biol. 8:231-237. Furthermore, primary APCs, like DCs, express six HLA class I alleles and concomitantly present a greater diversity of HLA-peptide complexes. Cytotoxic T lymphocytes of other HLA-peptide specificities are therefore stimulated. In contrast, AAPCs express a single HLA class I molecule efficiently loaded with the relevant peptide.

Vigorous CTL responses were induced against two peptides expressed in melanoma, one derived from the MART-1 and the other from the gp 100 antigen. After two rounds of T cell stimulation, specific CTLs were induced in three out of three donors for MART-1 and two out of three for gp100. These findings are concordant with studies in melanoma patients and normal donors, suggesting that MART-1 elicits a greater immune response than gp100. Spagnoli et al. (1995) Int. J. Cancer 64:309-315; Rivoltini et al. (1996) J. Immunol. 156:3882-3891; and Kawakami et al. (1997) Int. Rev. Immunol. 14:173-192. These results demonstrate that AAPCs can induce strong responses against autoantigens and suggest that they do not only recall primed CTLs-as is the case for the flu response-but also activate naive T cells present at a very low frequency in the peripheral blood of healthy donors.

T cells induced by AAPCs against autoantigens specifically kill tumor cells that over-express these antigens in an HLA class I-restricted manner. This strongly suggests that AAPCs may be used to expand CTLs for clinical purposes. Artificial APCs are stably transduced and thus obviate the need to generate autologous primary cells to effectively induce populations of antigen-specific T cells for each patient. Artificial APCs can easily be generated for different MHC-peptide combinations, and could be modified to stimulate T helper cells if MHC class I-peptide complexes are expressed. Additional costimulatory and/or adhesion molecules may further augment their capacity to promote the expansion of antigen-specific T cell populations.

Currently, virally infected B cells or DCs are used to generate T cells for adoptive cell therapies. Riddell and Greenberg (1995); Rooney et al. (1998); O'Reilly et al. (1998); Brenner et al. (1998) Vox Sang. 2:87-90; and Heslop et al. (1996) Nat. Med. 2:551-555. Transduced mouse fibroblasts provide an alternative cellular system effective in activating B lymphoma cells (Schultze et al. (1997)), restimulating genetically modified T cells (Krause et al. (1998) J. Exp. Med. 188:619-626; and Gong et al. (1999) Neoplasia 1:123-127), or activating and expanding human primary T cells as shown here. Viral vectors facilitate the generation of AAPCs for other HLA molecules and peptides, starting from other cell types if necessary. Artificial APCs are therefore versatile and useful to study T cell activation and to induce antigen-specific T cells for clinical purposes.

### EXAMPLE 11

### Diagnostic use of AAPCs and Loading AAPC with Exogenous Peptide

The experiment was designed to show two things.

1. AAPC cells expressing the flu peptide from a transduced minigene can be used as targets in an ELISpot assay; and

2. AAPCs that express HLA and co-stimulatory molecules, but no endogenous minigene, can be pulsed with exogenous peptide and used as stimulators in the ELISpot. This broadens the use of the cells in the assay to a large number of antigens, without the need for individual genetic engineering of each line.

Source of T cells for ELISpot assay. PBMC from a healthy A2-2.1 donor were stimulated in vitro 4-5 times with the flu matrix peptide, GLV. The T cells were frozen. A vial was defrosted on day 1, along with a vial of PBMC from the same donor. The PBMC were pulsed with 10 μg/ml peptide, irradiated, washed, and used to stimulate the T cells. Initially, 80-100 units/ml IL-2 were added to the cultures (added every 2 days). T cell cultures were maintained in the absence of IL-2 until day 14. IL-15 can also be used effectively instead of IL-2.

ELISpot assay. On day 12, a HA-Multiscreen plate (Millipore) was coated with mouse anti-h-IFN-γ mAb. On day 14, the plate was washed and wells blocked in complete media +10% FCS. CD8⁺ T cells (5×10⁶) were obtained from the T cell culture (day 1) by positive selection on Miltenyi beads (Miltenyi Biotec GmbH).

CD8⁺ T cells were plated at a concentration of 5×10⁴/well. Target cells (AAPCs) were irradiated at 10,000 RADS and added to wells at 5×10⁴/well. Where indicated, peptide was added at 10 μg/well, and PHA at 5 μg/ml.

All experimental conditions were done in duplicate and included; CD8 T cells+AAPC-flu (AAPC transduced with flu minigene), CD8 T cells+AAPC+flu peptide (no minigene), CD8 T cells+AAPC without peptide; each class of AAPCs without CD8 T cells, T cells alone and T cells stimulated with the non-specific polyclonal activator PHA.

Cells were incubated at 37° C., 5% CO₂ for 20 hours. On day 17, cells were washed from the plate and secreted, captured IFN-γ were detected using biotin-conjugated anti-human IFN-γ and developed with reagents in the Vectastain kit. Spots were counted using an automated Zeiss Axioplan 2 microscope and MPC4 microscope control processor and analyzed using KS ELISpot software (Zeiss).

All data are reported as averages of two wells. There were less then 12 spots per well in all controls (AAPC alone, CD8 T cells alone, CD8 T cells plus AAPC in the absence of added peptide or transduced minigene). PHA stimulation gave 357 spots/well, CD8 T cells+AAPC+flu peptide gave 89 spots. The ELISpot reader could not count the spots in the AAPC-flu wells, there were too many spots. There were an estimated 1000 spots/well and the graphed data reflect that estimate. FIG. 9.

### EXAMPLE 12

### Tetrameric Complexes Allow Detection of Specific CTLs by Flow Cytometry

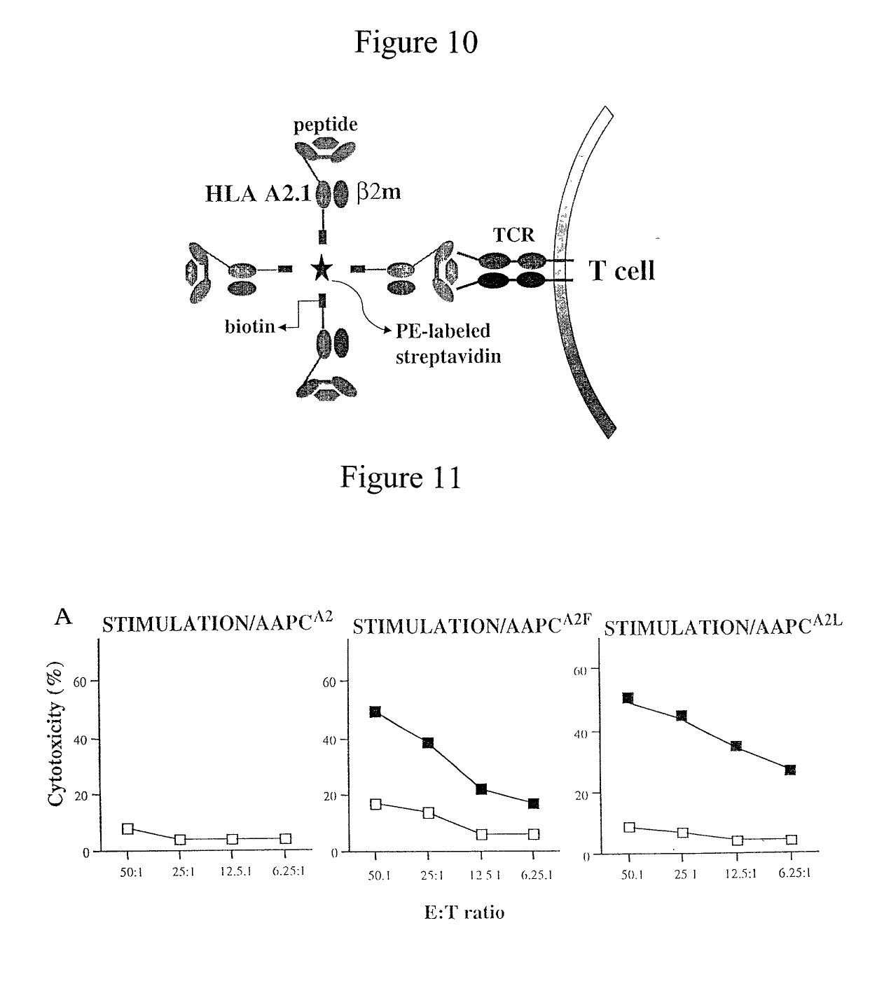

HLA A2.1/β2-microglobulin/peptide tetramers were synthesized in vitro by the following method: 1) cloning of HLA A2.1 and β2-microglobulin cDNAs in a prokaryotic expression vector so that expression of t gene results in soluble HLA A2.1; 2) purification of soluble HLA A2.1 from inclusion bodies; 3) In vitro refolding of HLA A2.1⁺ P2-microglobulin and peptide by dilution; 4) Biotinylation; 5) Fractionation of the correctly refolded monomer by FPLC (size exclusion column); 6) Tetramerization with PE-labeled streptavidin; and 7) Staining and identification of tetramer-specific T cells by FACS. The molecule obtained is shown in FIG. 10. The use of the tetramer to detect specific CTLs is illustrated in Examples 13 and 14.

### EXAMPLE 13

### AAPCs Efficiently Stimulate LMP1.1 Cytotoxic T Cell Responses

The EBV-encoded latent membrane protein 1 (LMP 1) is consistently expressed in EBV-associated malignancies, and the peptide epitope YLLEMLWRL derived from LMP1 (LMP1.1) is presented in the context of the HLA class I molecule A2.1. Starting from peripheral blood T cells harvested from HLA A2.1⁺ donors, it was shown that the AAPCs consistently elicit strong stimulation of CTLs with HLA-restricted specific cytotoxic activity against the LMP1.1 peptide.

FIG. 11 shows the results obtained. In FIG. 11 A, CTLs from HLA A2.1⁺ donor were stimulated with HLA A2.1⁺ AAPCs without peptide (AAPCA2), expressing the flu peptide (AAPC^{A2F}), or expressing the LMP1.1 peptide (AAPC^{A2L}). Standard ⁵¹Cr release assays as described herein were performed using T2 cells as targets. Filled symbols correspond to target cells pulsed with the relevant peptide, open symbols to target cells pulsed with an irrelevant peptide. The Y axis shows the percentage of specific ⁵¹Cr release; the X axis shows the effector to target E:T ratios. In FIG. 11 B, CTLs, in the same experiment, were detected by flow cytometry using the tetramers described in Example 11. Cytotoxic T cells were stained with a Tricolor-labeled antibody against CD8 (Y axis), and PE-labeled tetramers (X axis).

### EXAMPLE 14

### AAPCs, but not Autologous EBV-transformed B Cells Stimulate LMP1.1-specific CTLs

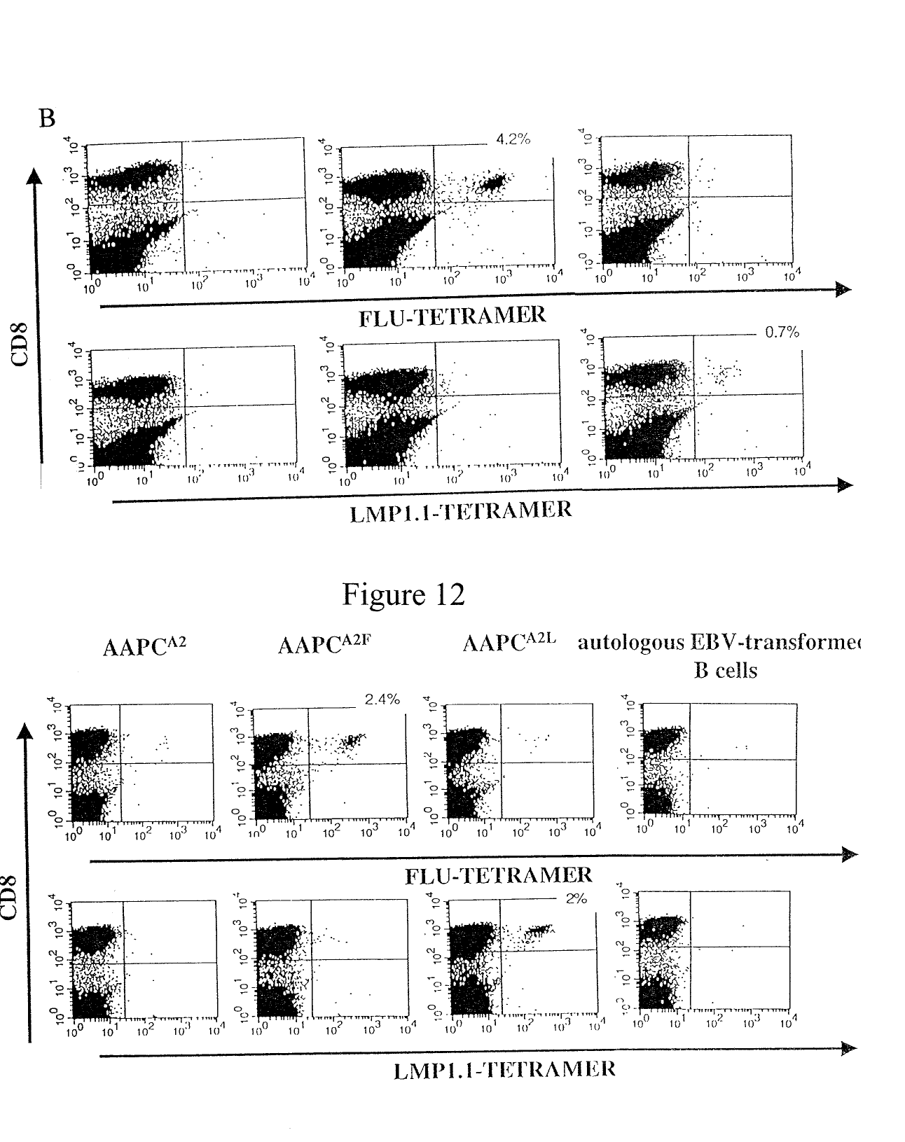

FIG. 12 shows the detection of specific CTLs by flow cytometry using the tetramers described in Example 11 after coculture of T cells from HLA A2.1⁺ donor with different AAPCs or autologous EBV-transformed B cells. CTLs from HLA A2.1+ donor were stimulated with AAPCs encoding the LMP1.1 peptide (AAPC^{A2L}) or autologous EBV-transformed B cells. AAPC^{A2} and AAPC^{A2F} were used as controls. Cytotoxic T cells were stained with a tricolor-labeled antibody against CD8 (Y-axis), and with PE-labeled tetramers (X-axis).

### EXAMPLE 15

### LMP1.1-specific CTLs Kill EBV-transformed B Cells and EBV-associated Lymphoma Cells in Vitro

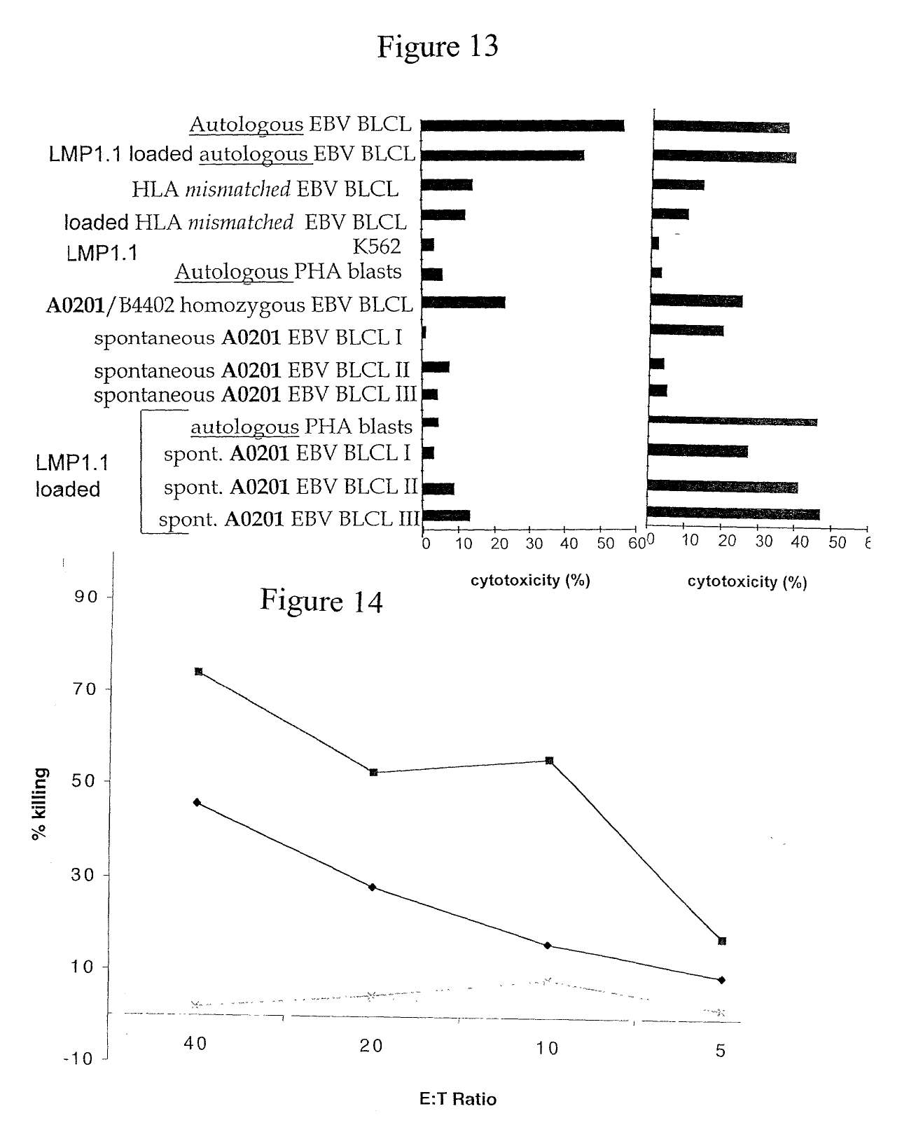

FIG. 13 shows the results of CTLs stimulated by autologous EBV-transformed B cells or AAPCs encoding the LMP1.1 peptide (AAPC) were compared in their abilities to kill different tumor cell lines. The effector to target ratio was 40:1.

The results obtained show that AAPCs efficiently stimulate LMP1.1-specific CTLs whereas, under identical conditions, autologous EBV-transformed B cells failed to do so. LMP1.1-specific CTLs thus have more utility in treating EBV-associated malignancies than autologous EBV-transformed B cells.

### EXAMPLE 16

### Expression of an Entire Protein by AAPCs Results in Peptide-specific T Cell Activation

AAPC were transfected with a vector expressing pp65, a CMV protein. Normal human T cells cultured with these AAPCs (as described in Example 11) are activated. T CTLs produced are specific for one of the pp65-derived peptides, E495. The results are shown in FIG. 14. These data demonstrate that the AAPC processed and presented pp65 in a T cell-specific manner.

### EXAMPLE 17

### Additional AAPC-induced CTL-activation Results

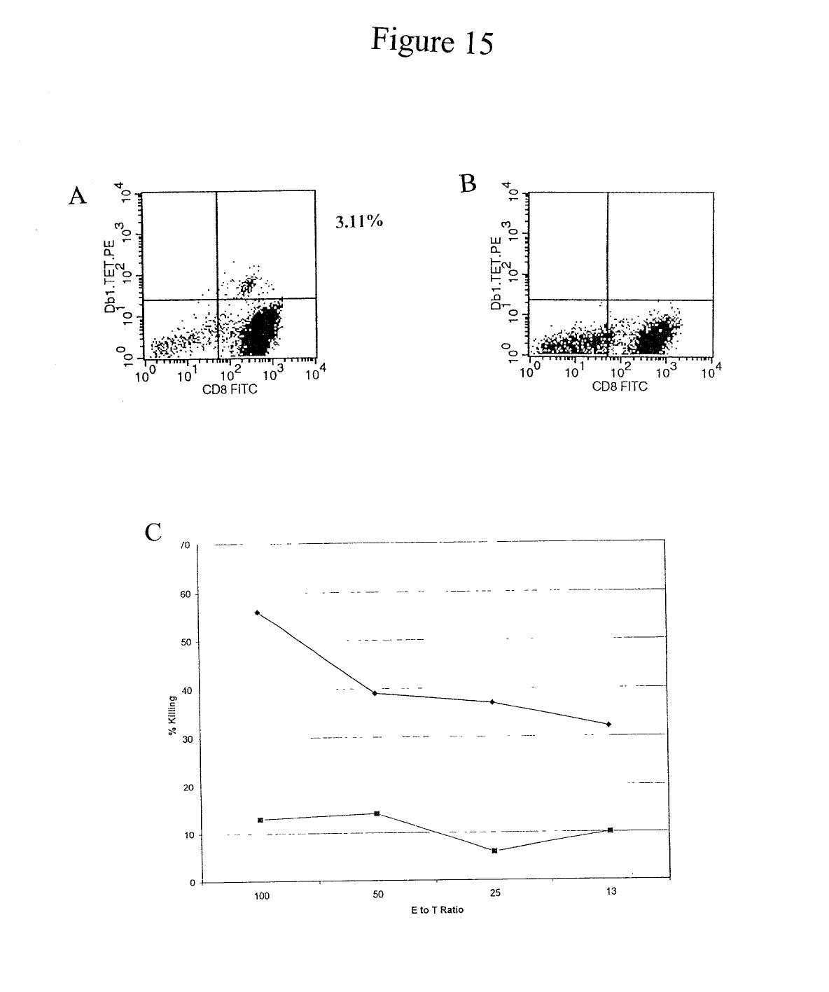

Additional results are shown in FIGS. 15-17 showing that the AAPCs of the present invention activate CTLs to Wilm's tumor, telomerase reverse transcriptase (hTERT) and a tumor cell line SKLY by hTERT-specific CTLs.

FIG. 15 shows the results from AAPCs constructed using HLA A2.1 restricted peptide Db126 (RMFPNAPYL, SEQ ID NO: 46). Tetramer staining was after 3 stimulations on AAPCs and ⁵¹Cr release was assayed after 4 stimulations on AAPCs. FIGS. 15A and B show, by WT1 (Db126) tetramer staining, (A) CTLs stimulated on WT1 (Db126) AAPCs and (B) the negative control, CTLs stimulated on WT1 (Wh187) AAPCs. FIG. 15C shows the results of the ⁵¹Cr release assay (T2 cells).

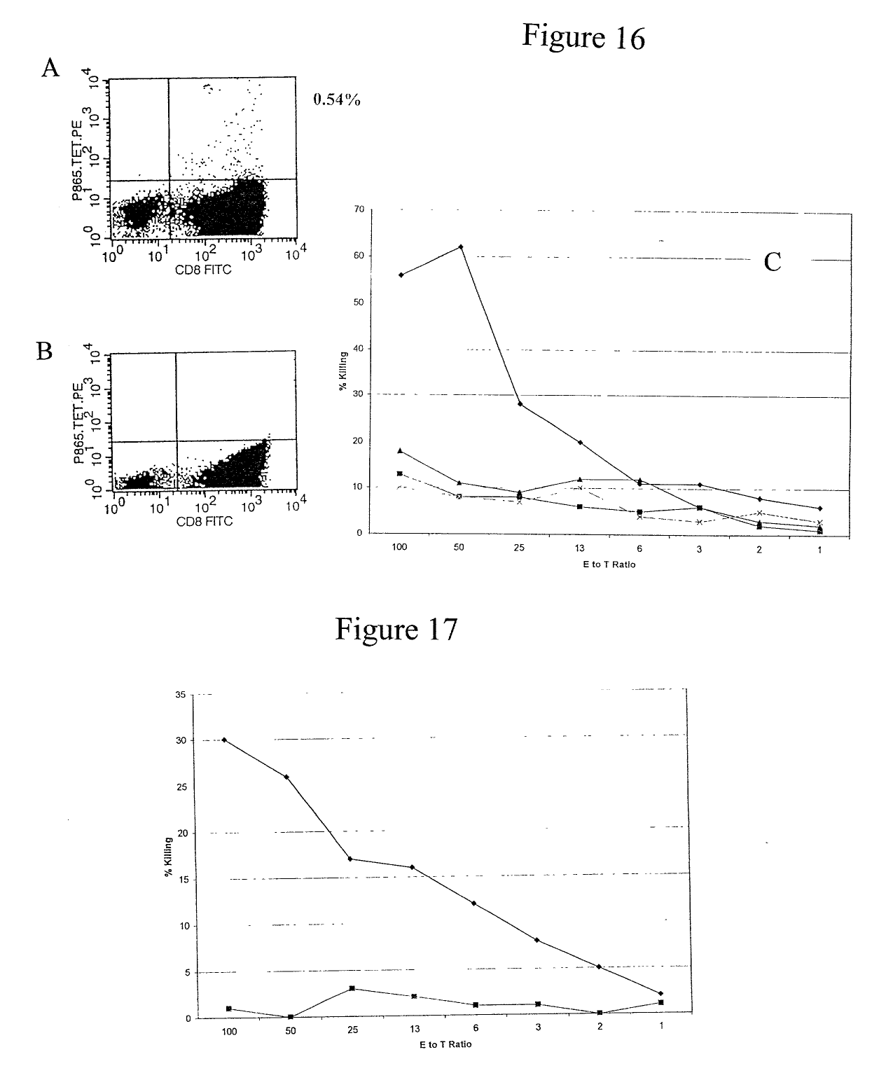

FIG. 16 shows the results from AAPCs constructed using HLA A2.1 restricted peptide P865 (RLVDDFLLV, SEQ ID NO: 47). Tetramer staining was after 4 stimulations on AAPCs and ⁵¹ Cr release was assayed after 4 stimulations on AAPCs. FIGS. 16A and B show, by hTERT (p865) tetramer staining, (A) CTLs stimulated on hTERT (p865) AAPCs and (B) the negative control, CTLs stimulated on empty AAPCs. FIG. 16C shows the results of the ⁵¹Cr release assay (T2 cells).

FIG. 17 shows results from AAPCs constructed using HLA A2.1 restricted peptide P865. Tetramer staining was after 4 stimulations on AAPCs and ⁵¹Cr release was assayed after 4 stimulations on AAPCs. Control CTLs were stimulated on HLA A2.1⁺ flu AAPCs. FIG. 17 shows the results of the ⁵¹Cr release assay (T2 cells).

All documents, publications and patent applications cited in this specification are incorporated herein by reference. Although the invention has been described in detail by way of illustration and example for purposes of clarity and understanding, certain modifications may be practiced. Therefore, the description and examples should not be construed as limiting the scope of the invention, which is delineated by the appended claims.

1. A parental artificial antigen presenting cell (AAPC) comprising a eukaryotic cell expressing β2-microglobulin and at least one exogenous accessory molecule.

2. An MHC-specific parental artificial antigen presenting cell (AAPC) comprising a eukaryotic cell expressing β2-microglobulin, at least one exogenous accessory molecule and a human leukocyte antigen (HLA) molecule of a single type.

3. An artificial antigen presenting cell (AAPC) comprising a eukaryotic cell expressing an antigen presenting complex comprising β2-microglobulin, at least one exogenous accessory molecule, a human leukocyte antigen (HLA) molecule of a single type and presenting at least one exogenous T cell-specific epitope.

4. The AAPC according to claim 1, 2 or 3 wherein the cell is selected from the group consisting of human, murine, rodentia, insect, or any other mammalian cells.

5. The AAPC according to claim 4, wherein the cell is human.

6. The AAPC according to claim 5, wherein the cell is autologous.

7. The AAPC according to claim 6, wherein the cell is non-autologous.

8. The AAPC according to claim 1, 2 or 3 wherein the cell is selected from the group consisting of fibroblast, T lymphocyte, tumor cell, transformed cell line, cell of hematopoietic origin, keratinocyte muscle cell or stromal cell.

9. The AAPC according to claim 8, wherein the cell is a fibroblast.

10. The AAPC according to claim 8, wherein the cell is a T lymphocyte.

11. The AAPC according to claim 1, 2 or 3, wherein the P2-microglobulin is endogenous.

12. The AAPC according to claim 1, 2 or 3, wherein the P2-microglobulin is exogenous.

13. The AAPC of claim 1, 2 or 3 wherein the β2-microglobulin is human β2-microglobulin.

14. The AAPC according to claim 1, 2 or 3 wherein the accessory molecule is selected from the group consisting of B7.1, B7.2, ICAM-1, LFA-3, CD40, CD40L, SLAM and 41BB ligand.

15. The AAPC according to claim 14, wherein the accessory molecule is B7.1.

16. The AAPC according to claim 14, wherein the accessory molecule is ICAM-1.

17. The AAPC according to claim 14, wherein the accessory molecules are B7.1 and ICAM-1.

18. The AAPC according to claim 2 or 3, wherein the HLA molecule is endogenous.

19. The AAPC according to claim 2 or 3, wherein the HLA molecule is exogenous.

20. The AAPC of claim 2 or 3 wherein the HLA molecule type is HLA-I.

21. The AAPC according to claim 20, wherein the HLA type is HLA-I and is selected from the group consisting of A2.1, or any other HLA A, B or C.

22. The AAPC according to claim 21, wherein the HLA molecule is A2.1.

23. The AAPC of claim 3 wherein the at least one exogenous T cell specific epitope comprises a plurality of antigens.

24. The AAPC according to claim 3, wherein the T cell-specific epitope is derived from a peptide specific to a tumor cell, a bacterial cell, a virus, a parasite or a normal human cell.

25. The AAPC according to claim 3, wherein the T cell-specific epitope is derived from a peptide that is a mutant or enhanced peptide derived from naturally occurring peptide specific to a tumor cell, a bacterial cell, a virus, a parasite or a normal human cell.

26. The AAPC according to claim 3, wherein the HLA is Al and the T cell specific epitope is selected from the group consisting of YTSDYFISY, YLDDPDLKY, IADMGHLKY, STDHIPILY, DSDGSFFLY, ATDFKFAMY, YTAVVPLVY and YTDYGGLIFNSY.

27. The AAPC according to claim 3, wherein the HLA is A2.1 and the T cell specific epitope is selected from the group consisting of LLDVPTAAV, SLLPAIVEL, YLLPAIVEI, MVDGTLLLL, YMNGTMSQV, MLLSVPLLLG, LLLDVPTAAV, LLLDVPTAAVQA, and VLFRGGPRGLLAVA.

28. The AAPC according to claim 3, wherein the HLA is A11 and the T cell specific epitope is selected from the group consisting of SVLNLVIVK, KVVNPLFEK, RTQNVLGEK, ASFDKAKLK, and ATAGDGXXELRK.

29. The AAPC according to claim 3, wherein the HLA is A24 and the T cell specific epitope is selected from the group consisting of KYPNEFFLL, YYEEQHPEL, AYVHMVTHF, and VYXKHPVSX.

30. The AAPC according to claim 3, wherein the HLA is A68.1 and the T cell specific epitope is selected from the group consisting of DVFRDPALK, KTGGPIYKR, and TVFDAKRLIGR.

31. The AAPC according to claim 3, wherein the HLA is B7 and the T cell specific epitope is selected from the group consisting of APRTVALTA, APRTLVLLL, APRPPPKPM, SPRYIFTML, RPKSNIVLL, LVMAPRTVL, APRTVALTAL, and AASKERSGVSL.

32. The AAPC according to claim 3, wherein the HLA is B27 and the T cell specific epitope is selected from the group consisting of RRIKEIVKK, GRIDKPILK, RRSKEITVR, RRVKEVVKK, and RRYQKSTWL.

33. The AAPC according to claim 3, wherein the T cell-specific epitope is selected from the group consisting of influenza matrix, Mart-1, gp100, LMP-1, Wt-1, acid phosphatase, Her-2/neu and telomerase.

34. The AAPC according to claim 1, 2 or 3, wherein the β2-microglobulin and the accessory molecule are expressed from genes introduced into the cell by a recombinant virus.

35. The AAPC according to claim 1, 2 or 3, wherein the β2-microglobulin and the accessory molecule and the HLA molecule are expressed from genes introduced into the cell by a recombinant virus.

36. The AAPC according to claim 3, wherein the β2-microglobulin and the accessory molecule, the HLA molecule and the T cell specific epitope are expressed from genes introduced into the cell by a recombinant virus.

37. The AAPC according to claim 3, wherein the β2-microglobulin and the accessory molecule, the HLA molecule and the protein encoding the T cell specific epitope are expressed from genes introduced into the cell by a recombinant virus.

38. The AAPC according to claim 3, wherein the P2-microglobulin and the accessory molecule and the HLA molecule are expressed from genes introduced into the cell by a recombinant virus and the T cell specific epitope is loaded onto the cell.

39. The AAPC according to claim 1, 2 or 3 further comprising mutations that decrease endogenous peptide transport.

40. The AAPC according to claim 3, wherein the antigen presenting complex is effective in activating cytotoxic T cells.

41. A method of activating cytotoxic T lymphocytes (CTLs) comprising the steps of:
a) obtaining an AAPC according to claim 3;
b) obtaining a suitable population of T lymphocytes;
c) contacting the AAPC with the population of T lymphocytes under conditions suitable for T lymphocyte activation; and
d) isolating the activated CTLs.

42. The method according to claim 41, further comprising the step of:
e) restimulating the CTLs by contacting a second time with the AAPC.

43. A composition comprising the CTLs obtained by the method according to claim 41.

44. A composition comprising the CTLs obtained by the method according to claim 42.

45. A method of treating a patient in need thereof comprising administering an effective amount of any one of the compositions according to claim 3.

46. A method of treating a patient in need thereof comprising administering an effective amount of the activated CTLs from the method of claim 41.

47. A method of treating a patient in need thereof comprising administering an effective amount of the activated CTLs from the method of claim 42.

48. A method of screening for accessory molecules comprising the steps of:
a) obtaining an AAPC according to claim 3;
b) expressing genes encoding potential accessory molecules in the AAPC;
c) obtaining a control AAPC that is the same as b) but does not express potential accessory molecules;
d) obtaining a suitable population of T lymphocytes;
e) contacting the T lymphocytes with the AAPC of b) under conditions suitable for activating T lymphocytes;
f) contacting the T lymphocytes with the AAPC of c) under conditions suitable for activating T lymphocytes; and
g) comparing the activation of the T lymphocytes from e) to the activation of the T lymphocytes from f);
wherein, if the activation of the T lymphocytes from e) is greater than that of the T lymphocytes of f), the potential accessory molecule is designated an accessory molecule.

49. A method of screening for T cell-specific antigens comprising the steps of:
a) obtaining an AAPC according to claim 2;
b) allowing the cells of a) to present potential T cell specific antigens;
c) obtaining a control AAPC that is the same as b) but does not present potential T cell specific antigens;
d) obtaining a suitable population of T lymphocytes;
e) contacting the T lymphocytes with the AAPC of b) under conditions suitable for activating T lymphocytes;
f) contacting the T lymphocytes with the AAPC of c) under conditions suitable for activating T lymphocytes; and
g) comparing the activation of the T lymphocytes from e) to the activation of the T lymphocytes from f);
wherein, if the activation of the T lymphocytes from e) is greater than that of the T lymphocytes of f), the potential T cell specific antigens is designated a T cell specific antigen.

50. The method according to claim 49, wherein the potential T cell specific epitope is expressed from a gene introduced into the cell by a recombinant virus.

51. The AAPC according to claim 49, wherein the potential T cell specific epitope is loaded onto the cell.

52. The AAPC according to claim 49, wherein the potential T cell specific epitope is produced by recombinatorial chemistry.

53. The AAPC according to claim 49, wherein the potential T cell specific epitope is produced by a phage display library.

54. A method of identifying, within a test population of cytotoxic T lymphocytes (CTLs), CTLs specifically activated against a known T cell antigen comprising the steps of:
a) obtaining an AAPC according to claim 3;
b) allowing the AAPC to present the known T cell antigen;
c) obtaining a control AAPC that is the same as b) but does not present the known T cell antigen;
d) obtaining the test population of T lymphocytes;
e) contacting the test population of T lymphocytes with the AAPC of b) under conditions suitable for activating T lymphocytes;
f) contacting the T lymphocytes with the AAPC of c) under conditions suitable for activating T lymphocytes; and
g) comparing the activation of the T lymphocytes from e) to the activation of the T lymphocytes from f);
wherein, if the activation of the T lymphocytes from e) is greater than that of the T lymphocytes of f), the potential accessory molecule is designated an accessory molecule.

55. The method according to claim 54, wherein the known T cell specific epitope is expressed from a gene introduced into the cell by a recombinant virus.

56. The AAPC according to claim 54, wherein the known T cell specific epitope is loaded onto the cell.

57. The method according to claim 54, wherein identification is by measuring cytokine secretion.

58. The method according to claim 57, wherein the cytokine is selected from the group consisting of IFN-γ, IL-4, IL-10 or TNF.

59. The method according to claim 57, wherein cytokine secretion is measured by immunologic methods.

60. The method according to claim 54, wherein activation is measured by a T cell surface marker.

61. The method according to claim 60, wherein the T cell surface marker is an activation marker.

62. The method according to claim 61, wherein the activation marker is selected from the group consisting of CD69, IL-2 receptor and IL-15 receptor.

63. The method according to claim 60, wherein the T cell surface marker is an effector molecule.

64. The method according to claim 63, wherein the effector molecule is selected from the group consisting of FasL and trail.

65. The method according to claim 54, further comprising the step of measuring the proportion of activated CTLs in the test population of CTLs.

66. The method according to claim 54 or 65, wherein the identifying or measuring is for diagnostic purposes.


SEQUENCE LISTING (49 sequences)

SEQ ID NO: 1 - protein, 9 aa, Homo sapiens
YTSDYFISY

SEQ ID NO: 2 - protein, 9 aa, Homo sapiens
YLDDPDLKY

SEQ ID NO: 3 - protein, 9 aa, Homo sapiens
IADMGHLKY

SEQ ID NO: 4 - protein, 9 aa, Homo sapiens
STDHIPILY

SEQ ID NO: 5 - protein, 9 aa, Homo sapiens
DSDGSFFLY

SEQ ID NO: 6 - protein, 9 aa, Homo sapiens
ATDFKFAMY

SEQ ID NO: 7 - protein, 9 aa, Homo sapiens
YTAVVPLVY

SEQ ID NO: 8 - protein, 12 aa, Homo sapiens
YTDYGGLIFNSY

SEQ ID NO: 9 - protein, 9 aa, Homo sapiens
LLDVPTAAV

SEQ ID NO: 10 - protein, 9 aa, Homo sapiens
SLLPAIVEL

SEQ ID NO: 11 - protein, 9 aa, Homo sapiens
YLLPAIVEI

SEQ ID NO: 12 - protein, 9 aa, Homo sapiens
MVDGTLLLL

SEQ ID NO: 13 - protein, 9 aa, Homo sapiens
YMNGTMSQV

SEQ ID NO: 14 - protein, 10 aa, Homo sapiens
MLLSVPLLLG

SEQ ID NO: 15 - protein, 10 aa, Homo sapiens
LLLDVPTAAV

SEQ ID NO: 16 - protein, 12 aa, Homo sapiens
LLLDVPTAAVQA

SEQ ID NO: 17 - protein, 14 aa, Homo sapiens
VLFRGGPRGLLAVA

SEQ ID NO: 18 - protein, 9 aa, Homo sapiens
SVLNLVIVK

SEQ ID NO: 19 - protein, 9 aa, Homo sapiens
KVVNPLFEK

SEQ ID NO: 20 - protein, 9 aa, Homo sapiens
RTQNVLGEK

SEQ ID NO: 21 - protein, 9 aa, Homo sapiens
ASFDKAKLK

SEQ ID NO: 22 - protein, 12 aa, Homo sapiens
  VARIANT (1)..(12): ′X′ can be any amino acid
ATAGDGXXELRK

SEQ ID NO: 23 - protein, 9 aa, Homo sapiens
KYPNEFFLL

SEQ ID NO: 24 - protein, 9 aa, Homo sapiens
YYEEQHPEL

SEQ ID NO: 25 - protein, 9 aa, Homo sapiens
AYVHMVTHF

SEQ ID NO: 26 - protein, 9 aa, Homo sapiens
  VARIANT (1)..(9): ′X′ can be any amino acid
VYXKHPVSX

SEQ ID NO: 27 - protein, 9 aa, Homo sapiens
DVFRDPALK

SEQ ID NO: 28 - protein, 9 aa, Homo sapiens
KTGGPIYKR

SEQ ID NO: 29 - protein, 11 aa, Homo sapiens
TVFDAKRLIGR

SEQ ID NO: 30 - protein, 9 aa, Homo sapiens
APRTVALTA

SEQ ID NO: 31 - protein, 9 aa, Homo sapiens
APRTLVLLL

SEQ ID NO: 32 - protein, 9 aa, Homo sapiens
APRPPPKPM

SEQ ID NO: 33 - protein, 9 aa, Homo sapiens
SPRYIFTML

SEQ ID NO: 34 - protein, 9 aa, Homo sapiens
RPKSNIVLL

SEQ ID NO: 35 - protein, 9 aa, Homo sapiens
LVMAPRTVL

SEQ ID NO: 36 - protein, 10 aa, Homo sapiens
APRTVALTAL

SEQ ID NO: 37 - protein, 11 aa, Homo sapiens
AASKERSGVSL

SEQ ID NO: 38 - protein, 9 aa, Homo sapiens
RRIKEIVKK

SEQ ID NO: 39 - protein, 9 aa, Homo sapiens
GRIDKPILK

SEQ ID NO: 40 - protein, 9 aa, Homo sapiens
RRSKEITVR

SEQ ID NO: 41 - protein, 9 aa, Homo sapiens
RRVKEVVKK

SEQ ID NO: 42 - protein, 9 aa, Homo sapiens
RRYQKSTWL

SEQ ID NO: 43 - protein, 9 aa, Artificial  Sequence
  PEPTIDE (1)..(9): influenza matrix protein-derived peptide.
GILGFVFTL

SEQ ID NO: 44 - protein, 9 aa, Artificial Sequence
  PEPTIDE (1)..(9): MART-1 protein-derived peptide
GILGFVFTL

SEQ ID NO: 45 - protein, 9 aa, Artificial Sequence
  PEPTIDE (1)..(9): gp-100 modified peptide
IMDQVPFSV

SEQ ID NO: 46 - protein, 9 aa, Homo sapiens
RMFPNAPYL

SEQ ID NO: 47 - protein, 9 aa, Homo sapiens
RLVDDFLLV

SEQ ID NO: 48 - protein, 9 aa, Homo sapiens
YLLEMLWRL

SEQ ID NO: 49 - protein, 9 aa, Homo sapiens
YLQQNWWTL